(19) United States
(12) Patent Application Publication
(10) Pub. No.: US 2013/0183662 A1
(43) Pub. Date: Jul. 18, 2013

(54) MEANS AND METHODS FOR IDENTIFYING AN INCREASED RISK OF SYSTEMIC LUPUS ERYTHEMATOSUS (SLE) PATIENTS FOR DEVELOPING RENAL MANIFESTATIONS

(71) Applicants: Arturo Zychlinsky, Berlin (DE); Abdul Hakkim Rahamathullah, Boston, MA (US); Volker Brinkmann, Berlin (DE); Reinhard Voll, Freiburg (DE)

(72) Inventors: Arturo Zychlinsky, Berlin (DE); Abdul Hakkim Rahamathullah, Boston, MA (US); Volker Brinkmann, Berlin (DE); Reinhard Voll, Freiburg (DE)

(73) Assignee: MAX-PLANCK-GESELLSCHAFT ZUR FORDERUNG DER WISSENSCHAFTEN E.V., Munich (DE)

(21) Appl. No.: 13/642,568

(22) PCT Filed: Apr. 21, 2011
(86) PCT No.: PCT/EP2011/056468

§ 371 Date: Apr. 2, 2013

(30) Foreign Application Priority Data
Apr. 22, 2010 (EP) 10160773.7

(51) Int. Cl. G01N 33/68

(52) U.S. Cl. CPC G01N 33/6875; G01N 33/6893
USPC 435/6.1; 435/7.92; 435/7.4; 435/287.2

(57) ABSTRACT

The present invention relates to means and methods for identifying an increased risk of systemic lupus erythematosus (SLE) patients for developing renal manifestations. The present invention further relates to polypeptides binding to one or more components of neutrophil extracellular traps (NET(s)) and to kits comprising components suitable to carry out the methods provided herein.

The present invention relates to means and methods for identifying an increased risk of systemic lupus erythematosus (SLE) patients for developing a disease or disorder, e.g., renal manifestations.

Systemic lupus erythematosus (SLE, or lupus) is a life-threatening, chronic and severe autoimmune disease that affects multiple tissues and organs (Rahman et al., N Engl J Med (2008), 358:929-939). SLE patients produce antibodies (Abs) against self, mainly against chromatin (Katzin et al., Cell (1996), 85:303-306) and often to neutrophil proteins like lactoferrin (Caccavo et al., Clin Rheimatol (2005), 24:381-387,), myeloperoxidase, proteinase-3 (Schnabel et al., Arthritis Rheum (1995), 38:633-637) and elastase (Nassberger et al., Lancet (1989), 1:509). Antibodies against these granular proteins are known as anti-neutrophil cytoplasmic antibodies (ANCA) (Bosch et al., Lancet (2006), 368:404-418). Antigens and antibodies form immune-complexes that can be deposited in kidneys and contribute to lupus nephritis, a frequent and dangerous organ manifestation in SLE. Infections can initiate flares and are a major cause for mortality in SLE patients (Zandman-Goddard et al., Clin Rev Allergy Immunol (2003), 25:29-40; Ruiz-Irastorza et al., Lancet (2001), 357:1027-1032).

Neutrophils are recruited to infection sites, where they release antimicrobial, extracellular traps (NET(s)) (Brinkmann et al., Science (2004), 303:1532-1535; Buchanan et al., Curr Biol (2006), 16:396-400; Clark et al., Nat Med (2007), 13:463-469). NET(s) are released during a novel form of cell death that requires reactive oxygen species (ROS) produced by the NADPH-oxidase complex (Fuchs et al., J Cell Biol (2007), 176:231-241). During this process, the nucleus decondensates and intracellular membranes disintegrate, thus allowing the mixing of nuclear and cytoplasmic components. Eventually, the plasma membrane ruptures to release NET(s) which contain chromatin and granule proteins (Brinkmann et al., Nat Rev Microbiol (2007), 5:577-582). Neutrophils of several species make NET(s) (Alghamdi et al., Biol Reprod (2005), 73:1174-1181; Lippolis et al., Vet Immunol Immunopathol (2006), 113:248-255; Palic et al., Dev Comp Immunol (2007), 31:805-816) and they are important in the immune defense against bacteria and fungi (Beiter et al., Curr Biol (2006), 16:401-407; Buchanan et al., Curr Biol (2006), 16:396-400; Clark et al., Nat Med (2997), 13:463-469; Urban et al., Cell Microbiol (2006), 8:668-676).

The pathological manifestations of SLE occur throughout the body and include inflammation, bland vasculopathy, vasculitis and immune complex deposition (Rahman et al., N Engl J Med (2008), 358:929-939). The kidneys can be severely affected including inflammation, cellular proliferation, abnormalities of the basement membrane and deposition of immune complexes.

Production of auto-antibodies against nuclear components is a hallmark of SLE. Specifically, auto-antibodies against double-stranded (ds) DNA often arise in lupus patients. 70% of SLE patients develop anti-ds-DNA antibodies, and those with elevated titres usually progress to clinical manifestations (Hahn, N Engl J Med (1998), 338: 1359-1368). It is thought that the major source of autoantigens in SLE are incompletely cleared apoptotic (Casciola-Rosen et al., J Exp Med (1994), 179: 1317-1330) and/or necrotic cells (Munoz et al., Rheumatology (Oxford) (2005), 44: 1101-1107). Lupus patients also have auto-antibodies against proteins associated with clearance, like Clq, SAP, apolipo- and mannose-binding proteins (Wilson et al., J Clin Invest (1985), 76: 182-190; Botto et al., Nat Genet (1998), 19: 56-59; Bickerstaff et al., Nat Med (1999), 5: 694-697). Inactivation of these proteins by formation of immune complexes hampers efficient clearance of cell debris and results in freely accessible cellular remnants that might serve as auto-antigens.

However, the mechanisms leading to SLE and sequelae are still not understood.

This technical problem has been solved by the embodiments provided herein and the solutions provided in the claims.

Accordingly, the present invention provides in vitro means and methods for identifying an increased risk of systemic lupus erythematosus (SLE) patients for developing renal manifestations.

In the present invention, it was surprisingly found that inefficient NET degradation is directly linked to the pathogenesis of SLE. Particularly, in the present invention it was found that impaired NET degradation correlates with renal manifestations. Furthermore, it was found that serum DNase1 is responsible for neutrophil extracellular trap (NET) degradation and that impaired NET degradation is due to the presence of either DNase1 inhibitors or anti-NET antibodies protecting NET(s) from nuclease degradation. The present invention further reveals that high anti-NET antibody titers and increased risk of developing, e.g., renal manifestations are correlated.

The present invention relates to the provision of NET(s) in methods such as assay tests and read-out systems for, in one embodiment, measuring degradation of NET(s) and the corresponding evaluation of a clinical or non-clinical status. In one aspect, the present invention relates to the provision of an assay test for determining the risk of developing or the severity of diseases or disorders by measuring the degradation level of NET(s), wherein a patient sample is used in such an assessment. In context of the present invention, an assay test for measuring the degradation level of NET(s) may be performed using, biological, immunological or biochemical assays like, inter alia, ELISA, EIA, RIA, immunoaffinity chromatography, fluorescence spectrometry or other assays as known in the art and as described and exemplified herein. In context with the present invention, the disease or disorder may be associated with the presence of non-degraded NET(s) and/or components thereof as described herein. In context with the present invention, for example, the NET(s) as described and provided herein and as suitable to be used in the methods described and provided herein may be derived from a healthy donor or may be prepared as described and exemplified herein.

Accordingly, the present invention relates to an in vitro method for determining the risk of developing or the severity of a disease or disorder of a patient, wherein said method comprises the steps of:

(a) assaying the degradation level of NET(s) with a patient sample; and
(b) comparing the result with the NET(s) degradation level with a control sample, wherein a NET degradation level with the patient sample compared to the control sample of less than 70% or a NET degradation level of degraded NET(s) compared to the total NET(s) of less than 70% indicates that the patient is at increased risk of developing or having the disease or disorder at a severe state. Said disease or disorder may be associated with the presence of non-degraded NET and/or one or more selected NET components as described herein, e.g., renal manifestations as described herein. As described herein, renal manifestations may be sequelae of SLE. Thus, in context with the methods described and provided herein, the patient whose degradation level of NET(s) is assayed may be an SLE patient. Methods for assaying the degradation level of NET(s) in context with the present invention are described and exemplified herein. For example, either the amount of one or more selected NET components present in non-degraded NET(s) or the amount of one or more NET components released from degraded NET(s) may be measured.

Particularly, the present invention provides an in vitro method for identifying a systemic lupus erythematosus (SLE) patient at increased risk of developing renal manifestations wherein the method comprises:
(a) obtaining a sample of body fluid from said patient;
(b) contacting said sample with neutrophil extracellular trap (NET);
(c) incubating said sample contacted with said NET, thereby allowing the NET to be degraded;
(d) isolating non-degraded NET and degraded NET separately;
(e) determining NET degradation level by measuring either
   (i) the amount of one or more selected NET components present in non-degraded NET; or
   (ii) the amount of one or more selected NET components released from degraded NET; and
(f) (α) comparing the result with a control where the NET were contacted with a sample of body fluid obtained from a healthy individual, or
   (β) comparing the ratio of degraded NET versus the total NET within one sample,
      wherein
(α) a NET degradation level of the patient sample compared to the control sample of less than 70% or
(β) a NET degradation level of degraded NET compared to the total NET of less than 70% indicates that the patient is at increased risk to develop renal manifestations.

There is a direct correlation between NET degradation and the presence of anti-NET antibodies in the sera of SLE patients. As the inventors of the present invention have found, patients with poor NET degradation have high levels of anti-NET antibodies compared to patients who degraded NET normally. The present invention therefore also provides a method to identify patients with high levels of non-degraded NET by measuring the levels of anti-NET antibodies. For this purpose, we developed a cell based NET-assay where whole NET are immobilized onto a 96-well plate. Then a serum sample is diluted 1:200 and added to the NET and incubated such as to allow anti-NET Abs to bind to the NET components. The Cy3 signal is then normalized to the Sytox signal, so the value is proportional to the amount of the NET in each well of the 96 well plate. The results are plotted as Relative Fluorescence Light Units (RFL). After washing with buffer, e.g., a Cy3 fluorescent labelled secondary antibody is added and incubated for 1 h and then washed. The plates are then read with a fluorometer at 518/590 nm wavelength (Fluorescent reading 1 or F1). Subsequently, 2 μM Sytox Green® is added and incubated for 5 min and then read at the fluorometer at 485/518 nm wavelength (Fluorescent reading 2 or F2). The result is calculated by using the formula (F1/F2)×100. The levels of anti-NET antibodies from normal healthy donors are considered negative. SLE patients' sera with high titers of anti-NET antibody (non-degrader) can be used as a positive control and SLE patients' sera with low levels of anti-NET Abs (degrader) as a negative control. Test values above the positive control are an indication of high levels of anti-NET antibodies which would predict high risk of lupus nephritis since these antibodies can hamper NET degradation and eventually lead to the deposition of NET-immune complexes that could get deposited in the kidney leading to inflammation and nephritis.

Furthermore, in line with the invention provided herein, several NET components have been detected and identified. To this effect, NET were partially degraded with MNase and then precipitated by acetone. The samples were then solubilised in 40 ml of 500 mM triethylammonium bicarbonate buffer pH 8.5 (TEAB) and reduced with 2 ml of 50 mM tris-(2-carboxyethyl)phosphine (TCEP) for 60 min at 60° C. After alkylation with 1 ml of 200 mM methyl methanethiosulfonate (MMTS) at RT for 10 mM, each sample was incubated over night at 37° C. with 10 ml of a 200 mg/ml trypsin solution, solubilised in 500 mM TEAB. The reaction was stopped with 1 ml of a 10% TFA solution to obtain a final concentration of TFA of app. 0.2%. The sample was centrifuged for 10 min at 138006 g and the supernatant used for LC/MS analysis. The samples were analyzed by bottom-up nano-C/MALDI-MS as described in detail in http://web.mpiib-berlin.mpg.de/cgi-bin/p dbs/lc/index.cgi. Proteins were digested with trypsin and the resulting peptides separated by nano-LC (Dionex). Peptides were fractioned (Probot microfraction collector, Dionex) and analyzed with a 4700 Proteomics Analyzer (Applied Biosystems) MALDI-TOF/TOF instrument. The criterion for the identification of a protein was a minimum number of 3 peptides fulfilling the Mascot homology criteria. Candidates with two peptides fulfilling these criteria were verified by checking the fragmentation rules, such as hypercleavage sites (Asp, Glu, Pro), the appearance of common immonium masses and mass losses. A protein was considered as being localized to NET only when found in at least two independent samples from different donors. Exceptions are MNDA, actinin and lysozyme C. MNDA and actinin were identified with one peptide in independent samples only, however, the peptide is unique to both proteins within the IPI-database. Presence of lysozyme C in NET was verified by immunoblotting. A list of the components is shown in Table 1. Histone proteins were identified as being the most abundant proteins.

In the method of the present invention, it is also envisaged that only one or more NET component is contacted and incubated with a body fluid sample. In this context, the degradation of this one or more NET component contacted and isolated with the body fluid sample is measured and the corresponding NET degradation level is determined Examples for said one or more NET component contacted and isolated with the body fluid sample whose degradation level is to be measured are (i) nucleosome complex which is composed of DNA, histone H2A, histone H2B, histone H3, histone H4, as well as (ii) DNA, dsDNA, histone H2A, histone H2B, histone H3, histone H4, as well as (iii) neutrophil proteins such as Neutrophil Elastase, S100A8, lactoferrin, azurocidin, cathepsin G, S100A9, myeloperoxidase, proteinase 3, actin, lysozyme C, catalase and/or any other protein listed in Table 1. Preferably, said NET component contacted and isolated with the body fluid sample whose degradation level is to be measured is DNA or Neutrophil Elastase. Subsequently, the NET degradation levels of the patient sample may be compared to the NET degradation level of the control sample in accordance with the method provided herein.

TABLE 1
List of identified NET components. Accession numbers refer to version
no. 120 of UniProtKB/SwissProt (http://141.14.152.84/cgi-bin/36525/
pdbs/lc/menu_frame.cgi), last modified on Mar. 2, 2010.

| | Accession |
|---|---|
| Protein | No. |
| ACTB Actin, cytoplasmic 1 | IPI00021439 |
| ACTG1 Actin, cytoplasmic 2 | IPI00794523 |
| ACTN1 actinin, alpha 1 isoform a | IPI00013508 |
| ACTA1 Actin, alpha skeletal muscle | IPI00021428 |
| ACTR3 Actin-related protein 3 | IPI00028091 |
| ACTR3B Isoform 1 of Actin-related protein 3B | IPI00892652 |
| AZU1 Azurocidin | IPI00022246 |
| CAT Catalase | IPI00465436 |
| CTSG Cathepsin G | IPI00028064 |
| DEFA1; LOC728358 Neutrophil defensin 1 | IPI00005721 |
| ELA2 Leukocyte elastase | IPI00027769 |
| SERPINB1 Leukocyte elastase inhibitor | IPI00027444 |
| ENO1 Isoform alpha-enolase of Alpha-enolase | IPI00465248 |
| LCP1 Plastin-2 | IPI00010471 |
| LTF Growth-inhibiting protein 12 | IPI00298860 |
| LYZ Lysozyme C | IPI00019038 |
| MNDA Myeloid cell nuclear differentiation antigen | IPI00013163 |
| MPO Isoform H17 of Myeloperoxidase | IPI00007244 |
| MYH9 Isoform 1 of Myosin-9 | IPI00019502 |
| PR3 Leukocyte proteinase 3 | IPI00027409 |
| TKT cDNA FLJ54957, highly similar to Transketolase | IPI00643920 |
| S100A8 Protein S100-A8 | IPI00007047 |
| S100A9 Protein S100-A9 | IPI00027462 |
| S100A12 Protein S100-A12 | IPI00218131 |
| ANXA1 Annexin A1 | IPI00218918 |
| ANXA3 Annexin A3 | IPI00024095 |
| ANXA4 annexin IV | IPI00793199 |
| ANXA5 Annexin A5 | IPI00329801 |
| ANXA6 annexin VI isoform 2 | IPI00002459 |
| ANXA11 Annexin A11 | IPI00414320 |
| HIST1H2AB; HIST1H2AE Histone H2A type 1-B/E | IPI00026272 |
| HIST1H2BL Histone H2B type 1-L | IPI00018534 |
| HIST1H4C; HIST1H4D; HIST2H4A; HIST1H4I; | IPI00453473 |
| HIST1H4F; HIST1H4B; HIST1H4A; HIST1H4H; HIS | |
| HIST2H3D; HIST2H3A; HIST2H3C Histone H3.2 | IPI00171611 |
| HIST2HBE Histone H2B type 2-E | IPI00003935 |
| H2AFV Histone H2A.V | IPI00018278 |
| H2AFY H2A histone family, member Y isoform 2 | IPI00059366 |
| CAMP Cathelicidin antimicrobial peptide precursor | IPI00292532 |
| MMP9 Matrix metalloproteinase-9 | IPI00027509 |
| GAPDH Glyceraldehyde-3-phosphate dehydrogenase | IPI00219018 |
| CEACAM8 Carcinoembryonic antigen-related cell | IPI00013972 |
| adhesion molecule 8 | |
| GPI Glucose-6-phosphate isomerase | IPI00027497 |
| GSTP1 Glutathione S-transferase P | IPI00219757 |
| HBA1; HBA2 Hemoglobin subunit alpha | IPI00410714 |
| HBB Hemoglobin subunit beta | IPI00654755 |
| HSPA1B; HSPA1A Heat shock 70 kDa protein 1 | IPI00304925 |
| ITGAM Integrin alpha-M | IPI00217987 |
| LCN2 Neutrophil gelatinase-associated lipocalin | IPI00299547 |
| MMP8 Neutrophil collagenase | IPI00027846 |
| MSN Moesin | IPI00219365 |
| MYL6; MYL6B Isoform Non-muscle of Myosin light | IPI00335168 |
| polypeptide 6 | |
| PFN1 Profilin-1 | IPI00216691 |
| PPIA Peptidyl-prolyl cis-trans isomerase A | IPI00419585 |
| PYGL Glycogen phosphorylase, liver form | IPI00783313 |
| RNASE3 Eosinophil cationic protein | IPI00025427 |
| PGLYRP1 Peptidoglycan recognition protein | IPI00163207 |
| CEACAM6 Carcinoembryonic antigen-related cell | IPI00027412 |
| adhesion molecule 6 | |
| ITGB2 Integrin beta | IPI00103356 |
| ALDOA Fructose-bisphosphate aldolase A | IPI00465439 |
| CALM1; CALM3; CALM2 Calmodulin | IPI00075248 |
| CAPG Macrophage-capping protein | IPR007122 |
| CFL1 Cofilin-1 | IPI00012011 |
| FLNA FLNA protein (Fragment) | IPI00302592 |
| G6PD Isoform Long of Glucose-6-phosphate 1- | IPI00216008 |
| dehydrogenase | |
| GNAI2 Isoform 2 of Guanine nucleotide-binding protein | IPI00328744 |
| G(i), alpha-2 subunit | |
| GSN Isoform 1 of Gelsolin | IPI00026314 |
| GSN Isoform 2 of Gelsolin | IPI00646773 |
| HK3 Hexokinase-3 | IPI00005118 |
| MRLC2 Myosin regulatory light chain MRLC2 | IPI00033494 |
| P4HB Protein disulfide-isomerase | IPI00010796 |
| PGD 6-phosphogluconate dehydrogenase, | IPI00219525 |
| decarboxylating | |
| PGK1 Phosphoglycerate kinase 1 | IPI00169383 |
| PKM2 Isoform M1 of Pyruvate kinase isozymes M1/M2 | IPI00220644 |
| TPI1 Isoform 1 of Triosephosphate isomerase | IPI00451401 |
| UBB; UBC; RPS27A ubiquitin and ribosomal protein S27a | IPI00179330 |
| precursor | |
| VASP Vasodilator-stimulated phosphoprotein | IPI00301058 |
| VIM Vimentin | IPI00418471 |
| YWHAZ 14-3-3 protein zeta/delta | IPI00021263 |

As used herein, the terms “NET” or “NET(s)” may relate to a single NET or to a plurality of two or more NET(s). In accordance with the method of the present invention, NET(s) can be obtained by methods known in the art (Fuchs et al., J Cell Biol (2007), 176: 231-241; Bianchi et al., Blood (2009) 114(13):2619-2622). In one embodiment, the NET used in the method presented herein are derived from neutrophils of a healthy donor. In this context, said neutrophils may be isolated from the blood of said donor. This can be conducted, for example, using the Percoll® gradient method (Aga et al., J Immunol (2002), 169: 898-905). Therein, peripheral venous blood of a healthy donor is drawn using heparin. Histopaque-1119® density gradient medium (Sigma-Aldrich) or another gradient made of dextran is then filled into a jar and the drawn peripheral venous blood is topped onto the density gradient medium. After centrifugation, the peripheral blood is separated into plasma (upper phase), peripheral blood mononuclear cells (PBMCs) (interphase), neutrophils and some red blood cells (RBCs) (lower phase) and RBCs (pellet). The neutrophil-containing layer is then collected, washed and supplemented with human serum albumin (HSA). After centrifugation, the supernatant is discarded, the pellet is resuspended and added to a gradient stock. This gradient stock can be prepared with sterile Percoll® (GE Healthcare Life Sciences) resulting in gradients of concentrations of 65%, 70%, 75%, 80% and 85% in accordance with the manufacturer's instructions. After a further centrifugation step, the interphase between 70% and 75% Percoll® layers is collected and added with PBS/HSA. After another centrifugation, the pellet is resuspended in RPMI-Hepes. Neutrophils can then be counted with a cell counter under a light microscope. Neutrophils may then be suspended in medium such as RPMI-Hepes at a concentration resulting in the desired neutrophil concentration. For example, the final neutrophil concentration is 10⁶ neutrophils per ml. For the method provided herein, the neutrophils can then be seeded onto tissue culture plates or on cover-slips and activated for NET formation. This activation of neutrophils for NET formation can be catalysed by different reagents, such as lipopolysaccharides (LPS), phorbol myristate acetate (PMA), and microbes such as bacteria or fungi, either alive or fixed. The activation of neutrophils for NET formation can be carried out at temperatures between 35° C. and 39° C., with or without CO₂ and over a period of 3 to 10 hours. Preferably, the activation of 10⁶ neutrophils per ml for NET formation is conducted at 37° C. and 5% CO₂ over night using 20 nM PMA (Sigma-Aldrich). Subsequently, micrococcal nuclease (MNase) may be added to activated and the mixture shortly incubated at 37° C. for about 10 minutes. The supernatant is then removed and sedimented for about 10 minutes at 200×g. The supernatant contains the NET(s).

Alternatively, in the method provided in the present invention, also artificial NET(s) may be used. For this purpose, chromatin can be isolated from a cell of tissue culture, for example HeLa cells or HEK cells and incubated in vitro with commercially available purified neutrophil proteins such as Myloperoxidase (MPO) or Neutrophil Elastase (NE). These molecules will interact because of electrostatic interactions.

Samples of body fluid which are deployed in the method provided herein are obtainable by methods known in the art. In accordance with the method of the present invention, examples for samples of body fluid which may be contacted with NET(s) are blood, plasma, serum, lymphatic fluid, cerebrospinal fluid, vaginal fluid, semen, sputum, broncho-alveolar lavage fluid, ascites, faeces, faeces extracts or urine. Preferably, the sample of body fluid is blood or serum.

According to the present invention, a sample of body fluid from an SLE patient whose risk of developing, e.g., renal manifestations is to be identified is contacted with NET. Subsequently, said sample of body fluid contacted with NET can then be incubated. Said incubation allows the NET to be degraded. According to the method provided herein, non-degraded or degraded NET(s) may then be isolated and the NET degradation level determined. Said determination of the NET degradation level can be carried out by measuring either (i) the amount of one or more selected NET components present in non-degraded NET or (ii) the amount of one or more selected NET components released from degraded NET. From the amount measured in (i) or (ii), respectively, the NET degradation level of the patient sample and the NET degradation level of a control sample can be calculated and compared to each other. In context of the NET degradation level of a patient sample, during the contacting step of the method provided herein, the NET has been contacted with a sample of body fluid of an SLE patient whose risk for developing, e.g., renal manifestations is to be identified. In context of the NET degradation of the control sample, during the contacting step of the method provided herein, the NET has been contacted with a sample of body fluid of a healthy donor. In one embodiment of the method provided herein, the NET which is contacted with the samples of body fluids may originate from the same healthy donor as the control sample of body fluid. Said one or more selected NET component which is measured in (i) or (ii) may be nucleosome complex as defined above, DNA, dsDNA, Histone H2A, Histone H2B, Histone H3, Histone H4, Neutrophil Elastase, S100A8, Lactoferrin, Azurocidin, Cathepsin G, S100A9, Myeloperoxidase, Proteinase 3, Actin, Lysozyme C, Catalase and/or any other protein listed in Table 1. Preferably, said one or more NET component is DNA or Neutrophil Elastase.

As mentioned above, the present invention relates to the provision of methods such as an assay test for determining the degradation level of NET(s). In the assay test provided herein, a biological sample of a patient or a biological sample to be tested is contacted and incubated with NET(s). It is also contemplated that such assays may be performed with only single components/parts of NET(s). Such parts or components of NET(s) may be at least 2, at least 3, at least 4, at least 5 or more single components of NET(s) as described herein. Subsequently, in context with the present invention, the degradation level of said NET(s) or NET parts/component(s) contacted and incubated with the patient sample or biological sample to be tested is determined and compared to the corresponding NET degradation level of NET(s) which are contacted with a control sample, for example, a sample of a healthy donor. A lower NET degradation of the NET(s) or components thereof contacted with the patient sample compared to the control sample is indicative for the incapability of the patient to degrade NET(s) and, thus, for the presence of non-degraded NET(s) in the patient. Accordingly, such a patient is considered to be at increased risk for developing or having a disease or disorder at a severe state, particularly a disease or disorder which is associated with the presence of non-degraded NET(s). In context of the method described and provided herein, a NET degradation level of the patient sample compared to the control sample of less than 70% or a NET degradation level of degraded NET compared to total NET of less than 70% is considered as indicative for an increased risk developing or having a disease or disorder at a severe state, particularly a disease or disorder which is associated with the presence of non-degraded NET(s). The NET(s) or parts/component(s) thereof used in the method of the present invention may be of any origin, e.g., they may be derived from a healthy donor or prepared as described and exemplified herein. The sample (i.e., the patient sample, the test sample and/or the control sample) contacted with NET(s) or parts/component(s) thereof is preferably a body fluid sample as further described herein, e.g., blood, plasma, serum, lymphatic fluid, cerebrospinal fluid, vaginal fluid, semen, sputum, broncho-alveolar lavage fluid, ascites, faeces or faeces extract. In the assay test provided herein, the degradation level of NET(s) may be determined, e.g., by either measuring the amount of one or more selected NET component(s) present in non-degraded NET(s), or the amount of one or more selected NET component(s) released from degraded NET(s). For measuring the degradation level of NET(s) in context with the present invention, bioassays as commonly known in the art and as also described and exemplified herein may be employed. For example, the method described and provided herein may be performed using a biological, immunological or biochemical assay like, inter alia, ELISA, EIA, RIA, immunaffinity chromatography, a fluorescence spectrometry or the like as described herein. In one embodiment, the degradation level of NET(s) may be performed by using an ELISA as also described and exemplified herein. Herein, in one embodiment, an ELISA is provided which is capable of specifically detecting NET(s) and, thus, which is suitable to assay the degradation level of NET(s). This may be based on using a first antibody that binds to, e.g., the nucleosome complex (comprising, e.g., DNA, histone 2A and histone 2B), and a second antibody that binds to, e.g., Neutrophil Elastase. Indeed, a sample which contains chromatin, but not NET(s), would be bound by the first antibody, but not by the second. Vice versa, a sample which contains neutrophil proteins such as Neutrophil Elastase would not be bound by the first antibody. Such an ELISA assay is also described and exemplified herein (cf., e.g., Example 18 herein below). In this context, a first antibody may also be selected to bind to other NET components (such as DNA or any of the core histones as further described herein), and a secondary antibody may also be selected to bind to another neutrophil protein (such as, e.g., MPO, S100A8, or Cathepsin G) as further described herein. Of course, in context of the method described herein, the first antibody and the second antibody may also be switched such that (in context of the above example) the first antibody would bind to, e.g., Neutrophil Elastase, and the second antibody would bind to the nucleosome complex.

If the amount of one or more selected NET components present in non-degraded NET(s) is measured in the method provided herein, a higher amount of NET components present in non-degraded NET(s) is considered as a lower NET degradation level. That is, when measuring the amount of NET components present in non-degraded NET, said amount of NET components present in non-degraded NET(s) and the corresponding NET degradation level are reciprocally proportional. In this context, the degradation level is calculated by formula I:

degradation level=amount of NET components present in degraded NET/amount of NET components present in non-degraded NET

If the amount of one or more selected NET components released from degraded NET is measured in the method provided herein, a higher amount of NET components released from degraded NET is considered as a higher NET degradation level. That is, when measuring the amount of NET components released from degraded NET, said amount of NET components released from degraded NET and the corresponding NET degradation level are proportional. In this context, the degradation level is calculated by formula II:

degradation level=amount of NET components released from degraded NET/amount of NET components released after DNAse1 treatment

To calculate the percentage of the NET degradation level of a patient sample compared to the NET degradation level of a control sample, formula III can be used in accordance with the present invention:

%NET degradation=(NET degradation level of patient sample/NET degradation level of control sample)×100

To calculate the ration of degraded NET versus the total NET, formula IV can be used in accordance with the present invention:

%NET degradation=(NET released in the supernatant/total NET)×100

In accordance with the present invention, the NET degradation of the patient sample relates to the amount of degraded/non-degraded NET which, during the contacting step of the inventive method provided herein, was contacted with a body fluid sample of, e.g., an SLE patient whose risk for developing a disease or disorder, e.g., renal manifestations is to be identified. The NET degradation of the control sample relates to the amount of degraded/non-degraded NET which, during the contacting step of the inventive method provided herein, was contacted with a body fluid sample of a healthy donor.

In context of the present invention, a NET degradation level of the patient sample less than 70%, more preferably less than 65%, more preferably less than 60%, more preferably less than 55% and most preferably less than 50% compared to the NET degradation level of a control sample or a NET degradation level of degraded NET compared to the total NET of less than 70% more preferably less than 65%, more preferably less than 60%, more preferably less than 55% and most preferably less than 50% indicates that the patient is at increased risk of developing or having a disease or disorder (e.g., associated with the presence of non-degraded NET(s)) at a severe state. For example, this may be an indication that the patient is at increased risk to develop renal manifestations.

In accordance with the method of the present invention, if the amount of more than one NET component is measured, the comparison between the NET degradation level of the patient sample and the control sample may be based on the comparison between the combined amounts of the measured NET components contacted with the patient sample and the combined amount of the measured NET components contacted with the control sample. Alternatively, the amount of each single measured NET component contacted with the patient sample or the control sample, respectively, may be compared separately.

In accordance with the invention provided herein, examples for renal manifestations are lupus nephritis, glomerulonephritis, and small vessel vasculitis.

In one embodiment of the present invention, the NET which is contacted with a sample of body fluid may be immobilized on a solid phase. The binding of said NET on said solid phase may be directly or indirectly using a monoclonal antibody that recognizes a conformational epitope formed by DNA, H2A and H2B (Monestier et al., Mol Immunol (1993), 30: 1069-1075). As used herein, “direct binding” of said NET on a solid surface means that the NET is bound onto a substrate. “Indirect binding” as used herein in this context means that a binding molecule, e.g., an antibody is bound onto a solid surface and the binding molecule, e.g., the antibody in turn binds the NET. Preferably, the binding of said NET on said solid phase is indirectly. The indirect binding of said NET on said solid phase may be via a polypeptide binding specifically to a NET component. Examples for such NET components are nucleosome complex as defined above, DNA, dsDNA, histone H2A, histone H2B, histone H3, and histone H4, as well as Neutrophil Elastase, S100A8, lactoferrin, azurocidin, cathepsin G, S100A9, myeloperoxidase, proteinase 3, actin, lysozyme C, catalase and/or any other protein listed in Table 1. Preferably, in the present invention, the polypeptide via said NET is bound to said solid surface specifically binds to the nucleosome complex comprising histone H2A, histone H2B and/or DNA. More preferably, the polypeptide via said NET is bound to said solid surface specifically binds to histone H2A, histone H2B and/or DNA. Alternatively, the polypeptide via said NET is bound to said solid surface specifically binds to Neutrophil Elastase. According to the method provided herein, the polypeptide via said NET is bound to said solid surface may be an antibody.

In the method provided herein, the NET degradation level may be determined by measuring one or more selected NET component present in non-degraded NET. For measuring one or more selected NET components present in non-degraded NET, several assays may be employed. In the inventive method, examples for assays for measuring one or more selected NET components present in non-degraded NET are immunoassays such as ELISA/EIA or RIA, immunoaffinity chromatography, or fluorescence spectrometry and other bioassays as known in the art and as described and exemplified herein. Preferably, the immunoassay employed in accordance with the present invention is ELISA/EIA. In this context, first a polypeptide specifically binding to a NET component may be coated onto a solid phase such as ELISA Maxisorp (Nunc). Such a coating of a polypeptide (e.g., an antibody) onto a solid surface can be carried out by methods known in the art. Also, the solid phase may be coated with streptavidin or avidin as known in the art and as also exemplified herein. In this case, the polypeptide specifically binding to a NET component (e.g., a capture antibody) may further be biotinylated as known in the art (e.g., covalently attaching biotin to the polypeptide) and as exemplified herein. This may increase sensitivity. With regard to the coating of the solid phase with a binding polypeptide such as a capture antibody, for example, first the capture antibody is coated by diluting P12-3 mouse monoclonal antibody (directed against, e.g., the nucleosome complex) in carbonate buffer (100 mM, pH 9.5-9.7) to a final concentration of 4 μg/ml and incubating over night at room temperature. Cavities are then emptied. Subsequently, a blocking step follows by adding 200 μl per well of blocking buffer (PBS+1% BSA+0.01% Tween 20) followed by incubating for 60 min at 37° C. Cavities are again emptied and stored at −80° C. Afterwards, the plate(s) are/is thawed and human fluid samples are added. For this purpose, samples are diluted in blocking buffer (1:10 in master plate) and 50 μl of blocking buffer is added to cavities of an ELISA plate. Then 50 ml of diluted sample from master plate (final dilution 1:20) is added and incubated for 60 min at 37° C. The plate is then washed 3 times with washing buffer. NET can then be detected by adding 100 μl rabbit anti-Neutrophil Elastase (Calbiochem 481001 in blocking buffer, final concentration 12 μg/ml) and incubation for 60 min at 37° C. The plate is then washed 3 times with washing buffer. Subsequently, a secondary antibody, e.g., 100 μl dk anti-rabbit coupled to POD (Jackson Immuno Research 711-035-152 1:500 in blocking buffer) is added and incubated for 30 min at 37° C. The plate is then washed 3 times with washing buffer. For signal intensification, 100 μl rabbit anti-POD coupled to POD (Sigma P-2026) 1:500 in blocking buffer is added and incubated for 30 min at 37° C. The plate is then washed 3 times with washing buffer. Finally, 00 μl substrate in substrate buffer (0.5 ml TMB 1 mg/ml in DMSO+4.5 ml 50 mM phosphate/citrate buffer+5 μl H₂O₂) is added and incubated for 10 min at 37° C. Extinction is read at 650 nm. Examples for said NET components are nucleosome complex as defined above, DNA, dsDNA, histone H2A, histone H2B, histone H3, and histone H4, as well as Neutrophil Elastase, S100A8, lactoferrin, azurocidin, cathepsin G, S100A9, myeloperoxidase, proteinase 3, actin, lysozyme C, catalase and/or any other protein listed in Table 1. Preferably, said polypeptide coated on a solid phase specifically binds to the nucleosome complex comprising histone H2A, histone H2B and/or DNA. More preferably, said polypeptide via said NET is bound to said solid surface specifically binds to nucleosome complex comprising histone H2A, histone H2B and/or DNA or to Neutrophil Elastase. Said polypeptide may be an antibody. In a specific example, PL2-3 mouse monoclonal antibody binding to histone H2A, histone H2B and DNA (Losman et al., J Immunol (1992), 148:1561-1569) is coated onto a solid phase, e.g. an ELISA plate such as Maxisorp (Nunc). NET obtainable by methods described herein may then be added to the solid phase coated with said polypeptide specifically binding to a NET component. Subsequently, the NET(s) are captured by said polypeptide coated onto the solid phase and can then be contacted with a body fluid sample obtained from an SLE patient (patient sample) or from a healthy donor (control sample). According to the present invention, said patient body fluid sample contacted with said NET captured by the polypeptide coated onto the solid phase may then be incubated, thereby allowing the NET to be degraded. In order to determine the NET degradation level in accordance with the present invention, the amount of one or more selected NET components present in non-degraded NET may be measured. In this context, after the incubation step of the method provided herein, NET components released from degraded NET may be washed away. Subsequently, a further polypeptide specifically binding to a NET component can be added after the incubation step of the method of the present invention. Preferably, the NET component which said polypeptide added after the incubation step of the present invention binds to is different from the NET component the polypeptide coated on the solid surface binds to. Said polypeptide which is added after the incubation step of the inventive method may specifically bind to nucleosome complex as define above, DNA, dsDNA, histone H2A, histone H2B, histone H3, Neutrophil Elastase, histone H4, S100A8, lactoferrin, azurocidin, cathepsin G, S100A9, myeloperoxidase, proteinase 3, actin, lysozyme C and/or catalase or any other NET component listed in Table 1. Preferably, said polypeptide which is added after the incubation step of the inventive method specifically binds to nucleosome complex comprising histone A, histone B and DNA or to Neutrophil Elastase. For example, said polypeptide added after the incubation step of the inventive method is an antibody specifically binding to Neutrophil Elastase. After addition of said polypeptide after the incubation step of the inventive method, a secondary antibody binding said polypeptide may be added. Preferably, said secondary antibody is coupled to an indicator compound such as peroxidase (POD), horseradish peroxidase (HRP), alkaline phosphatise (ALP), glucoseoxidase (GOX). By subsequent addition of an appropriate substrate corresponding to said compound coupled to said secondary antibody, the amount of non-degraded NET components bound by specific polypeptides binding to one or more NET components can be measured. For example, if the compound coupled to said secondary antibody is HRP, said appropriate corresponding substrate subsequently added in the method provided herein may be a chromogenic substrate such as 3,3′,5,5′-Tetramethylbenzidine (TMB) or 3,3′-Diaminobenzidine (DAB) or a chemiluminescent substrate such as 3-aminophthalate. As described above, in accordance with the method provided herein, the NET degradation level may then be deduced from the measured amount of one or more non-degraded NET component. Subsequently, the NET degradation level of the patient sample can then be compared to the NET degradation level of the control sample and the risk for developing a disease or disorder, e.g., renal manifestations be identified.

Alternatively, in the inventive method provided herein, the NET degradation level may be determined by measuring one or more selected component released from degraded NET. In this context, first NET(s) are contacted with a body fluid sample obtained from an SLE patient. Said patient sample contacted with said NET may then be incubated, thereby allowing the NET to be degraded. In order to determine the NET degradation level, the amount of one or more selected NET components released from degraded NET may be measured. Examples for such NET components are nucleosome complex as defined above, DNA, dsDNA, histone H2A, histone H2B, histone H3, and histone H4, as well as S100A8, lactoferrin, azurocidin, cathepsin G, S 100A9, myeloperoxidase, proteinase 3, actin, lysozyme C catalase and/or any other protein listed in Table 1. Preferably, the NET component released from degraded NET which is measured in accordance with the present invention is the nucleosome complex comprising histone H2A, histone H2B and/or DNA, or Neutrophil Elastase.

In order to measure the one or more NET component released from degraded NET(s) which was contacted and incubated with a sample of body fluid in the method provided herein, for example, an immunoassay such as ELISA/EIA, immunoaffinity chromatography, or fluorescence spectrometry and other bioassays as known in the art and as described and exemplified herein may be employed. In context of the present invention, for an ELISA, a polypeptide specifically binding to said one or more NET component released from degraded NET may be added in the inventive method as described herein. Subsequently, a secondary antibody binding to said polypeptide bound to the one or more NET components released from degraded NET may be added. Preferably, said secondary antibody is coupled to an indicator compound such as peroxidase (POD), horseradish peroxidase (HRP), alkaline phosphatase (ALP), glucoseoxidase (GOX). By subsequent addition of an appropriate substrate corresponding to said compound coupled to said secondary antibody, the amount of NET components released from degraded NET and bound to said polypeptide specifically binding to one or more NET components can be measured and the corresponding NET degradation level be deduced. By comparing the NET degradation level of the patient sample to that of the control sample, the risk for developing a disease or disorder, e.g., renal manifestations can be identified according to the inventive method provided herein.

In the context of method provided herein, the NET contacted with the sample of body fluid and incubated therewith may be degraded by nucleases contained in the body fluid sample during the incubation step. Hence, said NET component released from degraded NET which is measured in accordance with the present invention may be DNA, preferably dsDNA. In this case, after incubation of said NET with said sample of body fluid, a reagent which stops nuclease activity may be added, such as EDTA or EGTA. For measuring the amount of DNA released from degraded NET, said patient body fluid sample contacted with said NET and supplied with said nuclease activity-stopping reagent may be centrifuged and the supernatant containing the DNA released from degraded NET can be isolated. Subsequently, a DNA quantification reagent can be added. Such DNA quantification reagents may be, for example, DNA intercalating dyes. Such intercalating dyes are well known in the art and encompass Picogreen® (Invitrogen), Sytox® Green (Invitrogen), Ethidium Bromide or Propidium Iodide. The amount of DNA released from degraded NET of the patient body fluid sample can then be measured by fluorescence spectrometry (Fuchs et al., J Cell Biol (2007), 176:231-241). The resulting NET degradation level of the patient sample (=the amount of DNA released from degraded NET) can then be compared to the NET degradation level of the control sample in order to identify the risk for development of a disease or disorder, e.g., renal manifestations.

The present invention further relates to assessing NET degradation by quantifying the amount of one or several of the NET components with enzymatic activity by using specific substrates. For this purpose, Neutrophil Elastase and NET-DNA were isolated by incubating 500 mU/ml MNase for 10 min in the non-degraded NET. Total Neutrophil Elastase is measured from unstimulated neutrophils lysed with 0.02% Triton X-100 in 1 M NaCl. Neutrophil Elastase activity is quantified with 100 μM of the peptide substrate N-(Methoxysuccinyl)-Ala-Ala-Pro-Val 4-nitroanilide (Sigma) for 15 min at room temperature. Optical density is measured at 405 nm (Microplate reader EL800; BIO-TEK Instruments). The percentage of NET-bound Neutrophil Elastase can be calculated from the ratio of the values obtained from the supernatant of the degraded NET and the total Neutrophil Elastase from cell lysates. Alternatively, the ratio of Neutrophil Elastase in degraded versus non-degraded NET can be calculated by dividing the “Neutrophil Elastase in the degraded NET” over “Neutrophil Elastase in the non-degraded NET”.

Another example of NET component is Myeloperoxidase (MPO). Enzymatic activity of MPO in the supernatant can be determined by a colorimetric assay (Hess et al., J Immun (1999), 163: 4564-4573). In brief, naïve neutrophils are lysed with 0.02% Triton X100 in 1 M NaCl and the MPO in the cell lysate serves as the control. NET-associated MPO is present in the degraded NET(s). MPO and NET-DNA was isolated by incubating 500 mU/ml MNase for 10 min in the non-degraded NET(s). 20 μl of cell lysates are added to 100 μl of substrate buffer (50 ml of citrate-phosphate buffer, pH 5; 20 ml of 30% H₂O₂ (Sigma); 20 mg of orthophenylenediamine (Sigma)). The reaction is then incubated for 10 min at room temperature and stopped with H₂SO₄ and the optical density was measured at 490 nm (Microplate reader EL800, BIO-TEK Instruments). The percentage of MPO was calculated based on the value present in the supernatant of the degraded NET and compared to the total MPO in the cell lysates. This would imply the percent MPO in the degraded NET(s). The percent MPO can also be calculated for the non-degraded NET based on the control. Alternatively, the ratio of MPO in degraded versus non-degraded NET can be calculated by dividing the MPO in the degraded/MPO in the non-degraded NET.

The present invention further relates to methods for detecting NET(s) in samples of body fluid. An example for a method for detecting NET(s) in samples of body fluid is an immunoassay such as ELISA/EIA. In this context, in the inventive method a solid phase such as an ELISA plate, e.g., Maxisorp (Nunc) is coated with a polypeptide binding to one or more NET components. Examples for such NET components are nucleosome complex as defined above, DNA, dsDNA, histone H2A, histone H2B, histone H3, and histone H4, as well as Neutrophil Elastase, S100A8, lactoferrin, azurocidin, cathepsin G, S100A9, myeloperoxidase, proteinase 3, actin, lysozyme C, catalase and/or any other protein listed in Table 1. Preferably, said polypeptide specifically binds to the nucleosome complex comprising histone H2A, histone H2B and/or DNA or Neutrophil Elastase. More preferably, said polypeptide via said NET is bound to said solid surface specifically binds to nucleosome complex comprising histone H2A, histone H2B and/or DNA or to Neutrophil Elastase. The polypeptide may be an antibody. In a specific example, PL2-3 mouse monoclonal antibody binding to histone H2A, histone H2B and DNA (Losman et al., J Immunol (1992), 148:1561-1569) is coated onto a solid phase such as an ELISA plate, e.g., Maxisorp (Nunc). Subsequently, a sample of body fluid of patient is added to the solid phase coated with said polypeptide specifically binding to one or more NET components and the NET is captured by said polypeptide onto the solid surface. For detection of NET contained in said sample of body fluid, in the method described herein a further polypeptide specifically binding to one or more NET components is added to the NET captured onto said solid phase. Said further polypeptide added to the NET captured onto said solid phase may bind to nucleosome complex as defined above, DNA, dsDNA, histone H2A, histone H2B, histone H3, and histone H4, as well as Neutrophil Elastase, S100A8, lactoferrin, azurocidin, cathepsin G, S100A9, myeloperoxidase, proteinase 3, actin, lysozyme C, catalase and/or any other protein listed in Table 1. Preferably, said further polypeptide added to the NET captured onto said solid phase binds to a different NET component than said polypeptide coated onto the solid phase. For example, said further polypeptide specifically binds to the nucleosome complex comprising histone H2A, histone H2B and/or DNA or Neutrophil Elastase. In a specific example, said further polypeptide specifically binds to Neutrophil Elastase. In the method provided herein, after addition of said further polypeptide added to the NET captured onto said solid phase, a secondary antibody binding said further polypeptide is added. Preferably, said secondary antibody is coupled to an indicator compound such as peroxidase (POD), horseradish peroxidase (HRP), alkaline phosphatise (ALP), glucoseoxidase (GOX). Optionally, a further antibody binding said indicator compound may be added for signal intensification. By subsequent addition of an appropriate substrate corresponding to said compound coupled to said secondary antibody, NET bound onto the solid surface by specific polypeptides binding to one or more NET components can be detected. For example, if the compound coupled to said secondary antibody is HRP, said appropriate corresponding substrate subsequently added in the method provided herein may be a chromogenic substrate such as 3,3′,5,5′-Tetramethylbenzidine (TMB) or 3,3′-Diaminobenzidine (DAB) or a chemiluminescent substrate such as 3-aminophthalate. Subsequently, the amount of detected NET can be measured by methods known in the art such as ELISA.

The present invention further relates to polypeptides binding to one or more NET components. Preferably, said binding to one or more NET components is specific. For example, in context with the method provided herein, these polypeptides may be employed for capturing NET onto a solid surface. In another example, these polypeptides may be used for binding one or more NET components (i) present in non-degraded NET or (ii) released from degraded NET. Preferably, said polypeptides are antibodies. The term “antibody” is used herein in the broadest sense and specifically encompasses intact monoclonal antibodies, polyclonal antibodies, multispecific antibodies (e.g., bispecific antibodies) formed from at least two intact antibodies, and antibody fragments, so long as they exhibit the desired biological activity. Also human and humanized as well as CDR-grafted antibodies are comprised.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be constructed as requiring production of the antibody by any particular method. For example, the monoclonal antibodies to be used in accordance with the present invention may be made by the hybridoma method first described by Kohler, G. et al., Nature 256 (1975) 495, or may be made by recombinant DNA methods (see, e.g., U.S. Pat. No. 4,816,567). “Antibody fragments” comprise a portion of an intact antibody. In context of this invention, antibodies specifically recognize one or more NET components obtainable by methods described herein.

The term “antibody” is herein used in the broadest sense and includes, but is not limited to, monoclonal and polyclonal antibodies, multispecific antibodies (e.g., bispecific antibodies), chimeric antibodies, CDR grafted antibodies, humanized antibodies, camelized antibodies, single chain antibodies and antibody fragments and fragment constructs, e.g., F(ab′)₂ fragments, Fab-fragments, Fv-fragments, single chain Fv-fragments (scFvs), bispecific scFvs, diabodies, single domain antibodies (dAbs) and minibodies, which exhibit the desired biological activity, in particular, specific binding to one or more of NET components as described herein.

The present invention further relates to kits comprising components suitable to carry out the methods provided herein. Such kits may comprise polypeptides binding to one or more NET components. Such polypeptides may be polyclonal or, preferably, monoclonal antibodies or fragments thereof binding to one or more NET components. Further comprised in the kits provided herein may be solid phases such as ELISA plates, e.g., Maxisorp (Nunc). Preferably, these solid phases are coated with one or more polypeptides provided herein binding to one or more NET components. Examples for such NET components are nucleosome complex as defined above, DNA, dsDNA, histone H2A, histone H2B, histone H3, Neutrophil Elastase, histone H4, S100A8, lactoferrin, azurocidin, cathepsin G, S100A9, myeloperoxidase, proteinase 3, actin, lysozyme C, catalase and/or any other Protein listed in Table 1. Preferably, the NET component which said polypeptide is binding to is nucleosome complex comprising histone H2A, histone H2B and/or DNA or Neutrophil Elastase. Optionally, the kit of the present invention may further comprise one or more NET components or a whole NET obtainable by methods described herein. These one or more NET components or the whole NET may be immobilized on a solid surface, either directly or indirectly. When bound indirectly to a solid surface, this bondage my be via a polypeptide as described herein binding to one or more NET components. The kit provided herein may further comprise DNase (e.g., DNase1), MNase or any other protein or enzyme able to degrade NET(s). Further comprised by the herein described kit may be serum of healthy donors as a control or any other suitable control sample of a healthy donor which is comparable to the patient sample which is to be tested. Also comprised may be secondary antibodies suitable for detection of binding of said polypeptide to the one or more nucleosome component as well as markers (e.g., HRP (horseradish peroxidase)) linked to the secondary antibody and chemical substrates such as POD chromogenic substrate. The kit to be prepared in the context of the present invention may further comprise or be provided with (an) instruction manual(s). For example, said instruction manual(s) may guide the skilled person (how) to identify, e.g., an SLE patient at increased risk of developing a disease or disorder, e.g., renal manifestations in accordance with the method of the present invention provided herein. Particularly, said instruction manual(s) may comprise guidance to use or apply the herein provided methods or uses. The kit to be prepared in context of this invention may further comprise substances/chemicals and/or equipment suitable/required for carrying out the methods and uses of this invention. For example, such substances/chemicals and/or equipment are solvents, diluents and/or buffers for stabilizing and/or storing said polypeptides provided herein required for carrying out the method of the present invention. In a specific embodiment, the kit described in the present invention may contain one or more ELISA plate(s) with NET(s) indirectly coated via anti-nucleosome antibodies (e.g., mouse monoclonal antibodies), MNase, serum of healthy donors, anti-Neutrophil Elastase antibody (NET detection antibody, rabbit polyconal antibody), one or more secondary antibodies (e.g., anti-rabbit secondary antibody coupled to POD), POD chromogenic substrate, and/or anti-POD antibody for intensification of the signal. In a particular embodiment, the kit may comprise an antibody binding to the nucleosome complex comprising Histone H2A, Histone H2B and DNA, an antibody binding to a NET component selected from the group consisting of Neutrophil Elastase, nucleosome complex, DNA, Histone H2A, Histone H2B, Histone H3, Histone H4, S100A8, Lactoferrin, Azurocidin, Cathepsin G, S100A9, Myeloperoxidase, Proteinase 3, Actin, Lysozyme C and Catalase, whole NET or one or more NET components selected from the group consisting of Neutrophil Elastase, nucleosome complex, DNA, Histone H2A, Histone H2B, Histone H3, Histone H4, Si 00A8, Lactoferrin, Azurocidin, Cathepsin G, S100A9, Myeloperoxidase, Proteinase 3, Actin, Lysozyme C and Catalasea and solvents, diluents. and/or buffers. The kit may further comprise an ELISA plate. The kit of claim 27, further comprising an ELISA plate.

The Figures show:

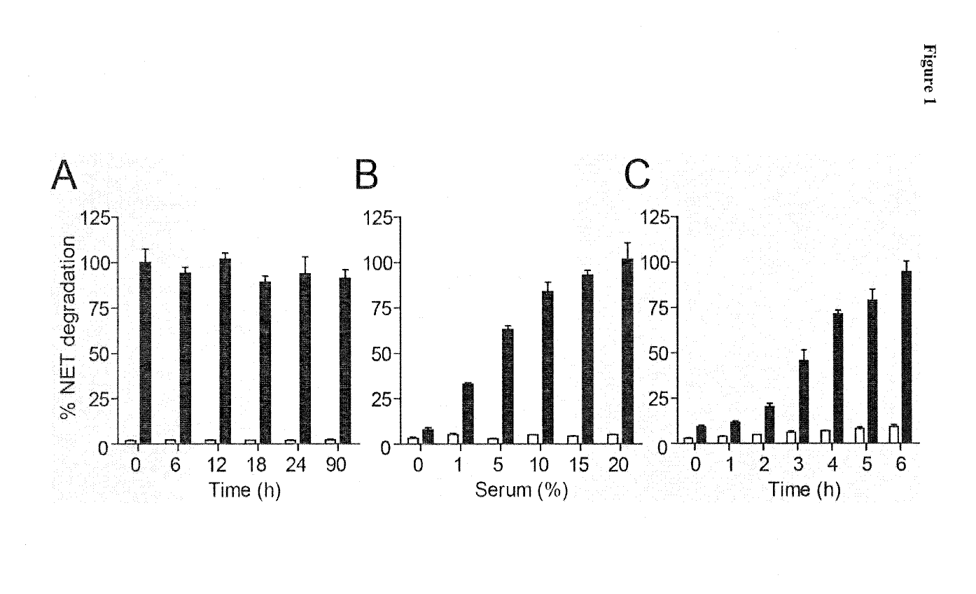

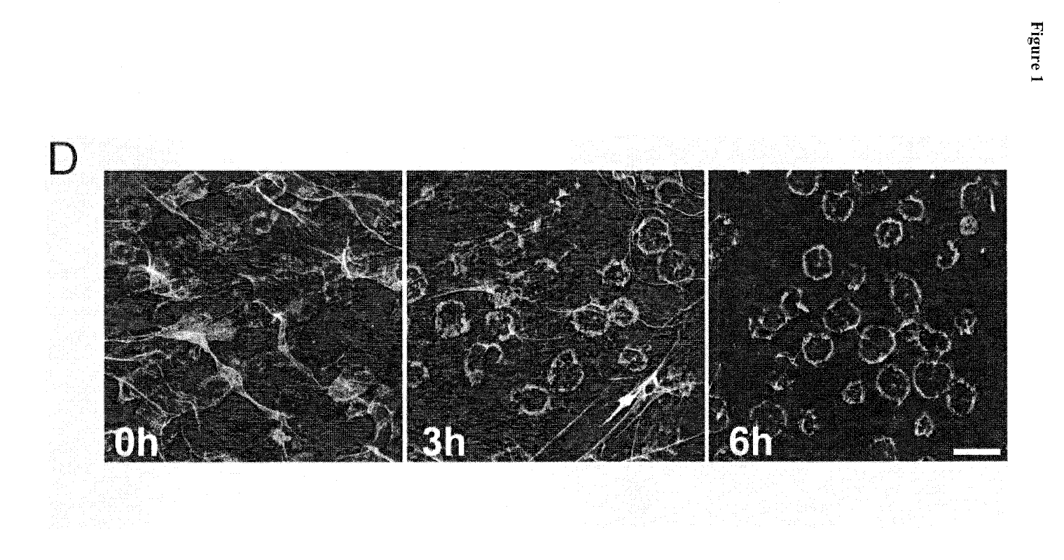

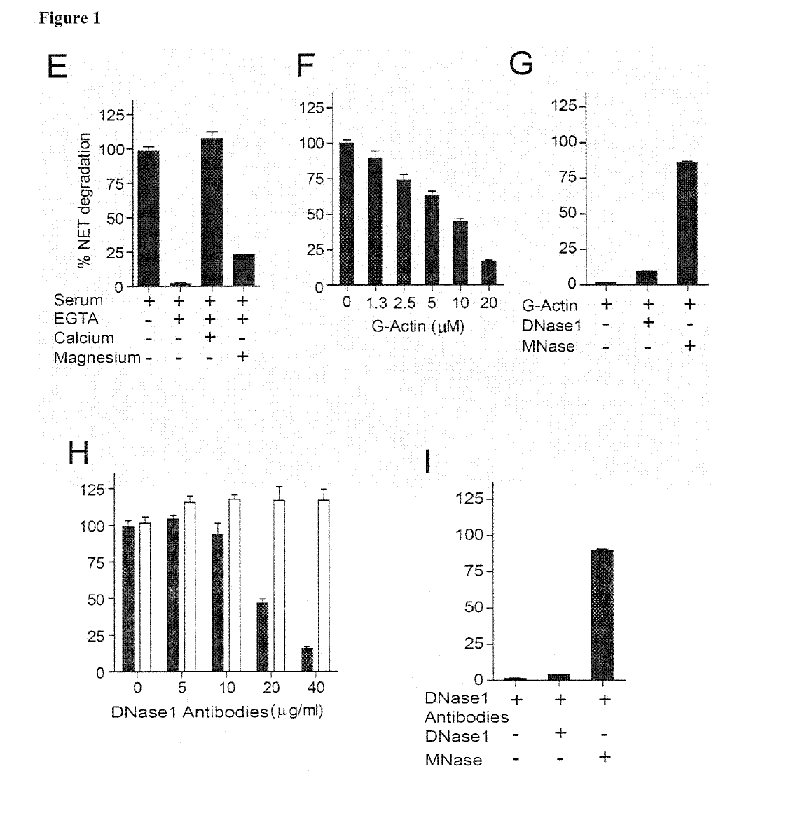

FIG. 1: Serum DNase1 Degrades NET

NET Degradation by Serum. Human neutrophils were activated to form NET and incubated in the indicated conditions before measuring the digested and released NET-DNA using the fluorescencent DNA dye Pico-Green. (A) In the absence of serum (open bars), NET(s) are stable for at least 90 hours in vitro. NET degradation with Microccocal Nuclease (MNase) for 10 min at each time point (black box) represents the total NET(s) recovered. (B) Serum-mediated degradation of NET(s) is concentration and (C) time-dependent when exposed to 10% serum, suggesting an enzymatic activity (open bars are medium controls). (D) Activated neutrophils that formed NET(s) were incubated in media with 10% serum for the indicated time points, fixed and immunostained for myeloperoxidase (green) and histones (red). DNA (blue) was stained with Draq5. Bar represents 10 μm. (E) Serum NET degradation requires calcium. NET(s) were incubated with 10% serum for 6 h. EGTA, a calcium chelator, inhibited degradation. Calcium, but not magnesium ions restored the NETdegrading activity. (F) Inhibition of NET degradation by G-actin, a specific inhibitor of DNase1, is dose dependent. NET(s) were incubated with 10% serum for 6 h in the presence of the indicated concentrations of G-actin and NET degradation was measured as described. (G) To control the specificity of G-actin for DNase1, NET(s) were incubated with 20 μM of G-actin with either commercially available purified DNase1 or MNase to digest the NET(s). G-actin blocked degradation by DNase1 but not MNase. (H) NET(s) were incubated with serum as described above and with the indicated concentrations of polyclonal anti-DNase1 (black bars) or irrelevant control antibodies (open bars). Inhibition of NET degradation by anti-DNase1 antibodies was specific and dose-dependent. (I) Anti-DNase1 antibodies are specific. NET(s) were incubated in the presence of 40 μg/ml of anti-DNase1 antibodies and incubated with purified DNase1 or MNase. The data shown are representative of experiment performed in triplicates and presented as a mean value±SD.

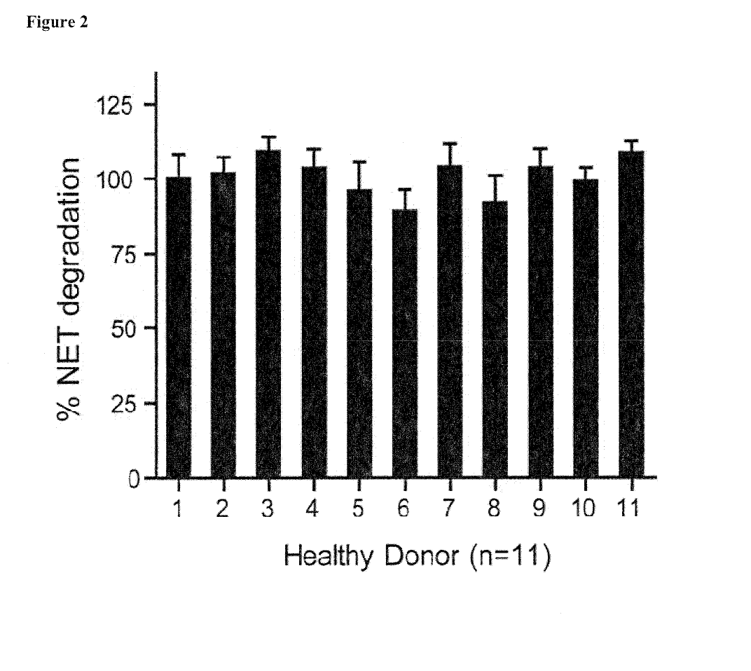

FIG. 2: Donor Variation in NET Degradation

NET made from neutrophils isolated from a healthy donor were exposed to sera from 11 different healthy donors. There was no significant donor variation in NET degradation. Data are mean of triplicates with standard deviation.

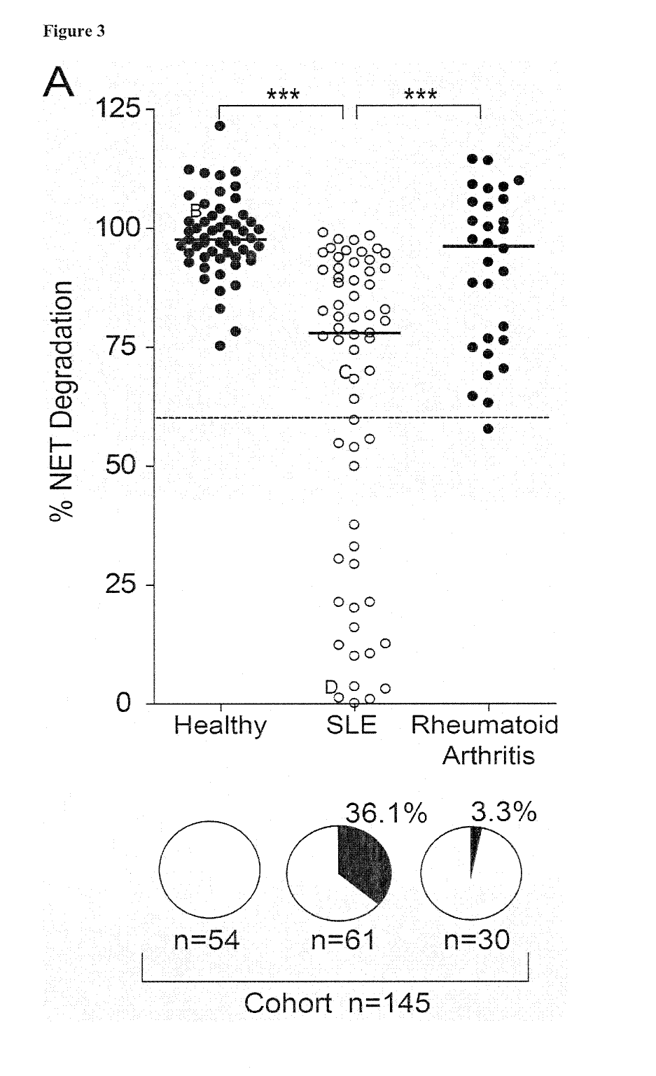

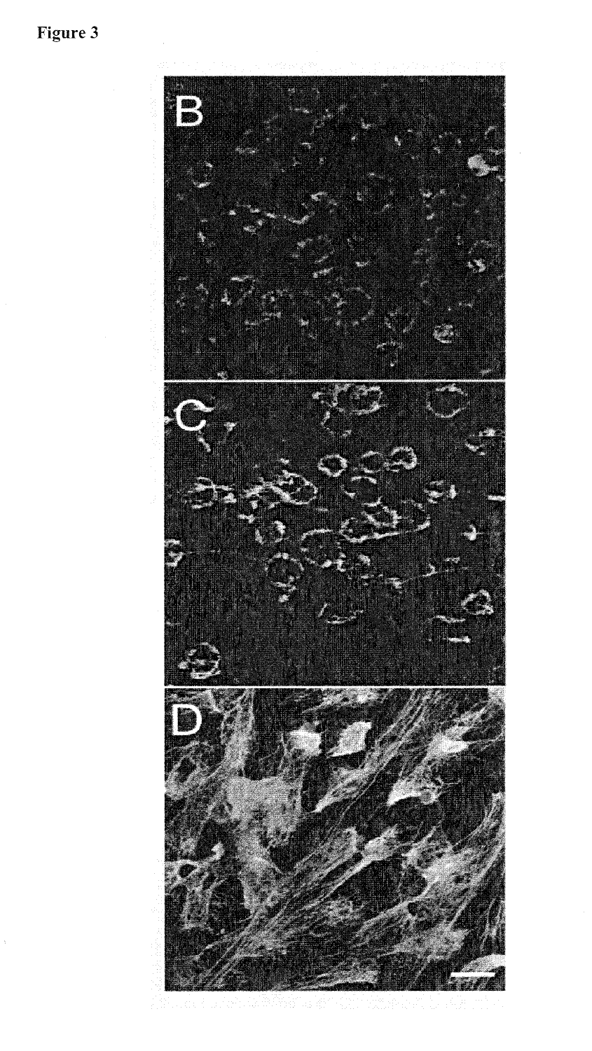

FIG. 3: NET degradation is impaired in a subset of SLE patients

(A) neutrophils isolated from healthy donors to make NET were activated, incubated with 10% sera from the cohort for 6 h and quantified NET degradation. The cohort is described in the methods section and in Table 2. Each circle corresponds to one individual donor. The samples are grouped into healthy donors, SLE and rheumatoid arthritis patients as indicated. One hundred percent NET degradation was determined using the serum from the healthy donor of the neutrophils. Sera that degrade at least 60% of the NET within 6 h was considered normal (horizontal line). Sera from all healthy donors (n=54, black circles) degraded NET(s) normally; 36.1% of SLE patient sera (n=61, open circles) and 3.3% of the RA sera (n=30, grey circles degraded NET(s) poorly. (***p<0.001, ns=non-significant using, Kruskal-Wallis test with Dunn's post-hoc comparisons). (B-D) NET(s) were exposed to representative sera (labeled B, C or D in panel A), fixed and immunostained for myeloperoxidase (green) and histones (red). DNA (blue) was stained with Draq5. Representative micrographs, show efficiency of NET degradation, with serum from a healthy donor (B), from a SLE patient that degraded NET(s) (C) and from a SLE patient that did not disassemble NET(s) (D). Bar=25 μm (B-D).

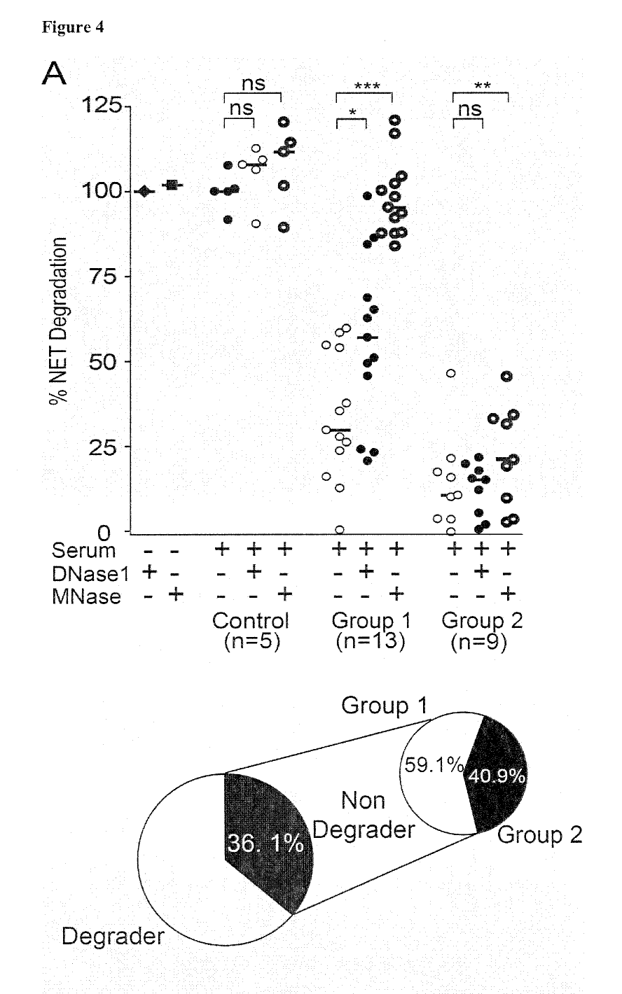

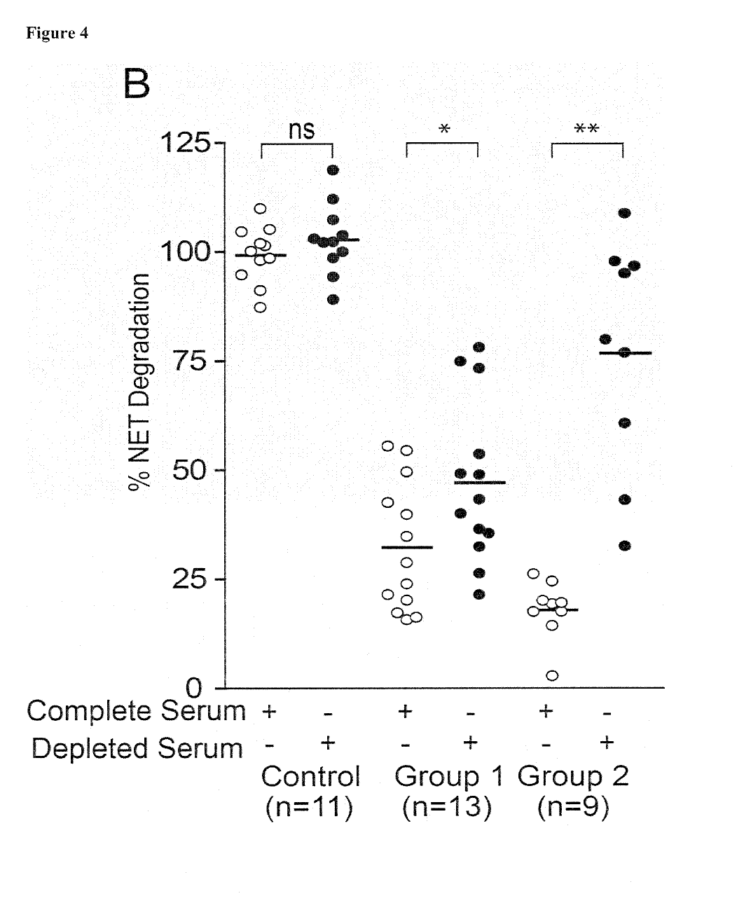

FIG. 4. Inhibitory mechanisms of NET degradation

(A) A subset of SLE sera contains DNase1 inhibitor(s). NET(s) incubated with sera from healthy donors (n=5) or SLE patients that did not degrade NET(s) (n=22) were spiked with exogenous DNase1 or MNase and then we quantified NET degradation. Degradation of NET(s) by healthy sera was unaffected by the addition of the exogenous nucleases. The SLE non-degrader sera fell into two groups: In Group 1, addition of MNase but not DNase1 fully restored NET degradation activity, suggesting the presence of specific DNase1 inhibitor(s). In Group 2, neither DNase1 nor MNase completely restored NET degradation, suggesting a mechanism of NET protection. (***p<0.001, *p<0.05, p>0.05, ns=non-significant compared by Friedman's test with Dunn's post-hoc comparison). The bar denotes the median of the group. Protecting antibodies impair NET degradation. (B) Sera from NET degraders and non-degraders were depleted of antibodies with protein A/G sepharose beads. The antibody-depleted sera were incubated with NET(s) for 6 h before quantification of NET degradation. Depletion of antibodies increased the NET degradation of sera from Group 1 only marginally. In contrast, sera from patients in Group 2 degraded NET efficiently after depletion. This indicates that sera from patients of Group 2 contain antibodies shielding NET(s) from degradation. (***p<0.0001, **p=0.0056, ns=non-significant using parametric paired t-test, since the data followed a Gaussian distribution). Each circle in panels A and B represents the activity of a single serum and is the value of the mean in an experiment performed in triplicates. The bars denote the mean of the group.

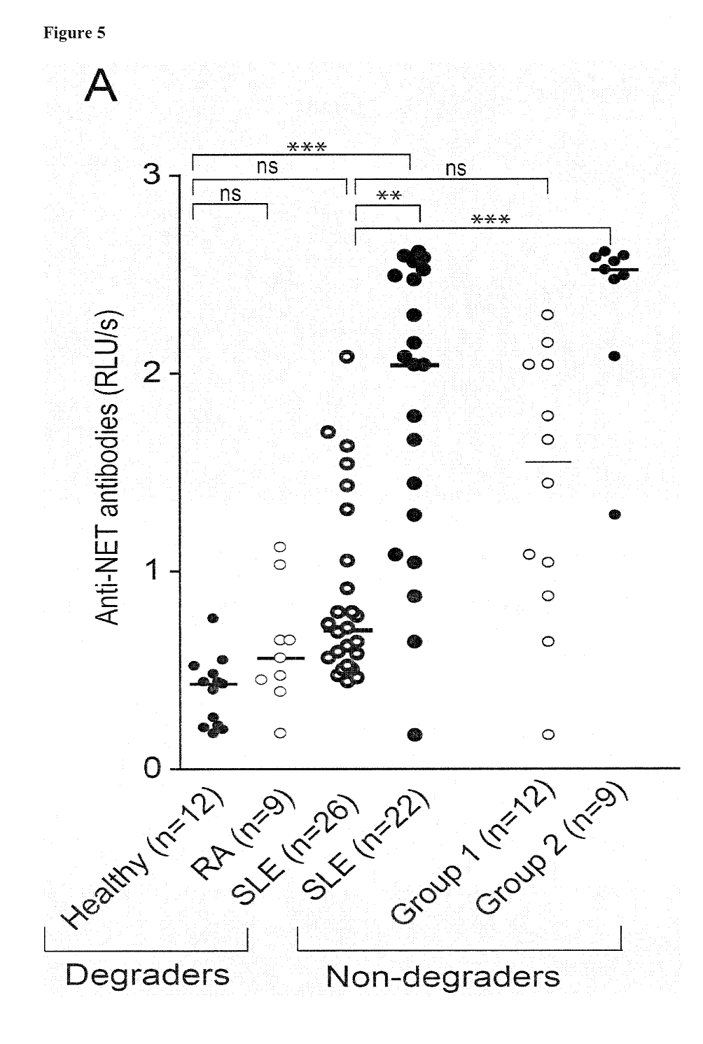

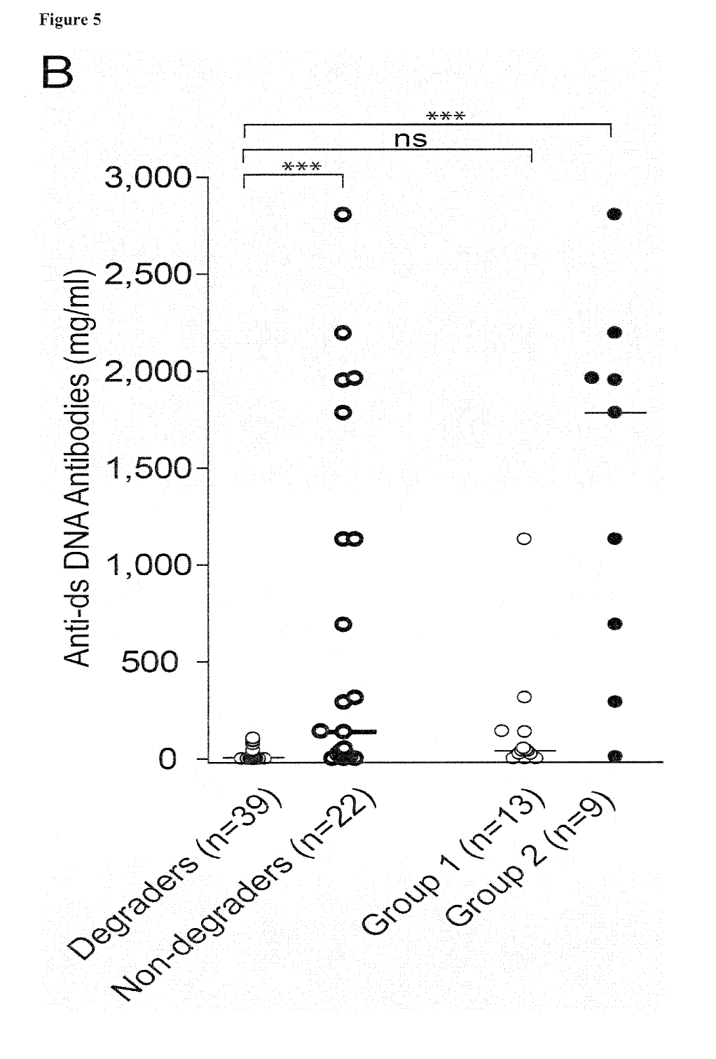

FIG. 5. Defective NET degradation correlates with high anti-NET Abs anti-dsDNAanti-nuclear antibody titers and increased risk of lupus nephritis

(A) Antibodies binding to NET(s) were quantified with a cell based NET-Assay, where NET are used as antigen, patient sera as primary antibodies and anti-human Cy3 coupled antibodies as secondary antibodies. Sera from healthy donors, from SLE degraders or from patients with other autoimmune diseases did not contain anti-NET antibodies. Most of the sera in Group 2 contained high levels of anti-NET antibodies. Sera in Group 1, however, were heterogeneous, but as a group, the concentrations of anti-NET antibodies were significantly higher than in the NET degraders. In A and B, each circle represents the activity of a single serum and is the mean of an experiment performed in triplicate. Bars show the median of the group. (B) The concentrations of anti-dsDNA antibodies were significantly higher in SLE nondegraders compared to degraders. (C) The titers of anti-nuclear antibodies detected by indirect immunofluorescence on fixed H[[p2 cells were significantly higher in non-degraders compared to the degraders. There is a significant difference between SLE degraders and Group 2 but not with Group 1. (***p<0.001, *p<0.05, ns p>0.05 using Kruskal Wallis test with post-hoc Dunn's multiple comparison test). Each circle in panel A, represents the mean of a triplicate experiment with patient serum. The bar denotes the median of the group. (D) We retrospectively analyzed the association between ineffective NET degradation with nephritis. There was a higher incidence of nephritis in SLE non-degraders than the degraders. Group 1 and Group 2 showed a significantly higher risk of nephritis when compared to NET degraders. The statistics for panel D is based on Fisher's exact test. The odds ratios with 95% confidence interval between non-degraders and degraders is 6.79 (2.108-21.86), with **p value of 0.0012; between degraders and Group 1 is 5.73 (1.457-22.52) with *p value of 0.0188, between degraders and Group 2 is 8.909 (1.596-49.74) with **p value of 0.0091.

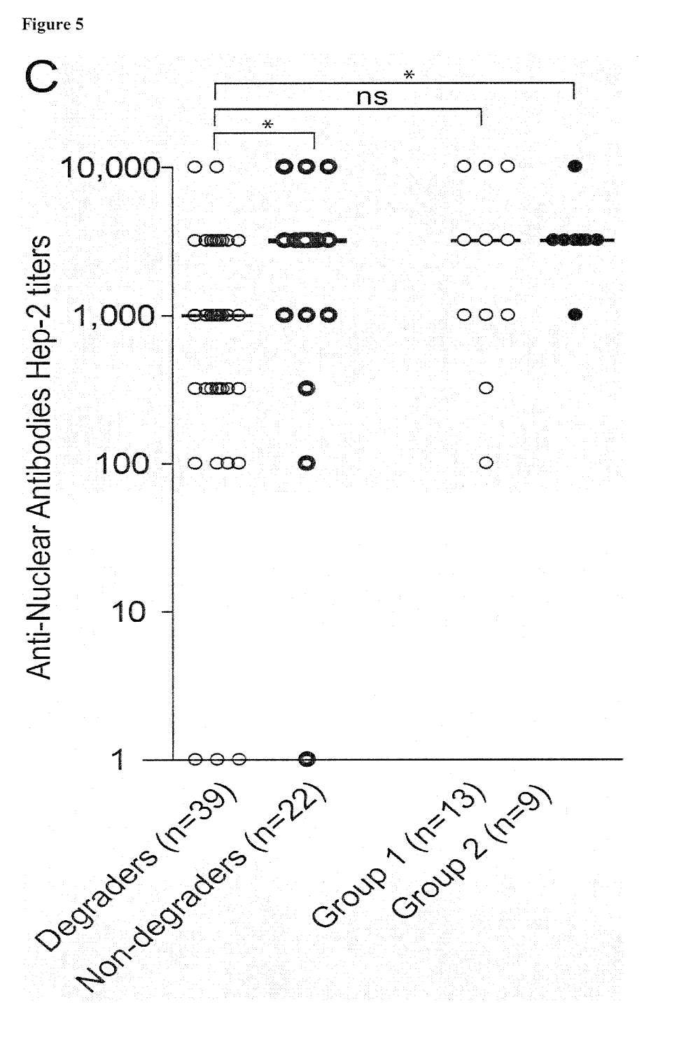

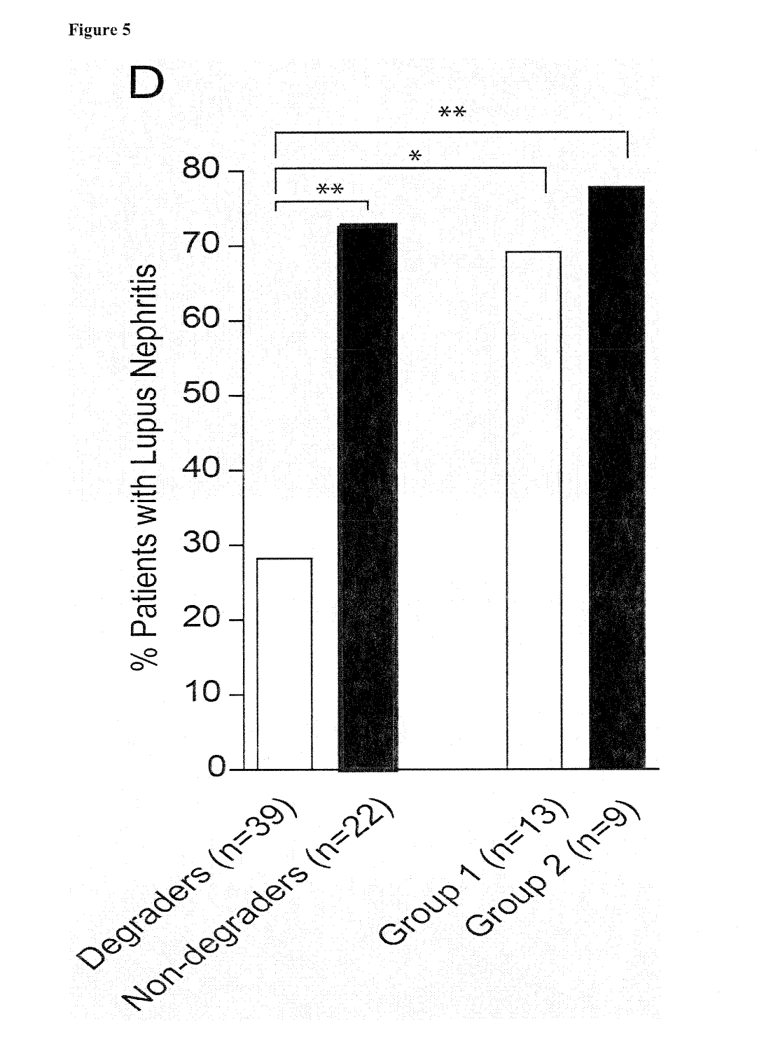

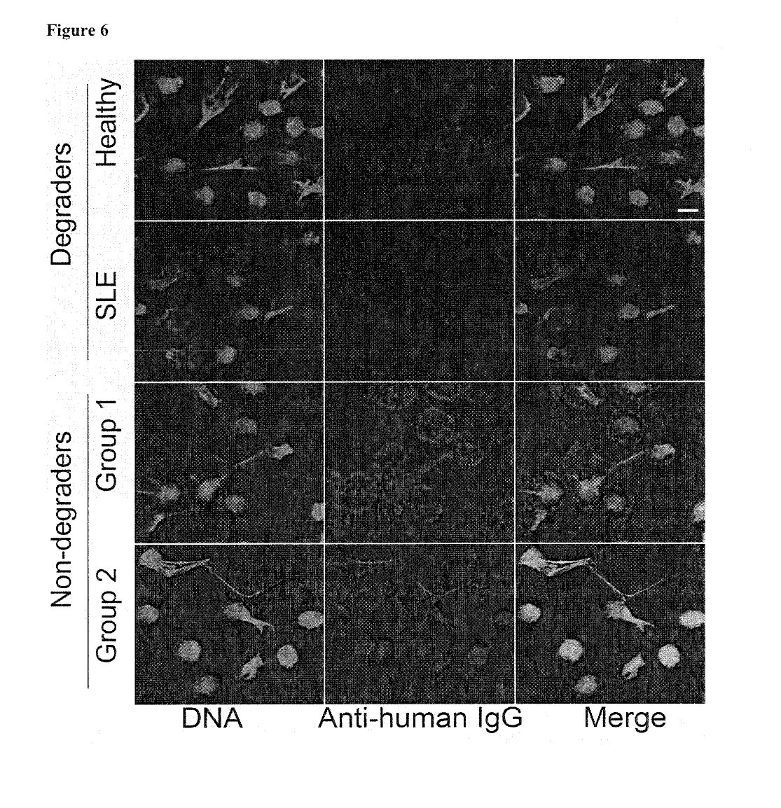

FIG. 6: Anti-NET Abs are elevated in non-degraders

Neutrophils were activated to make NET, incubated with sera from the indicated donors and then with anti-human secondary antibodies (red). DNA was stained with Draq 5 (blue) for contrast. Sera from non-degraders recognized the NET(s) in contrast to sera from healthy donors or from SLE degraders. Scale bars=10 μm.

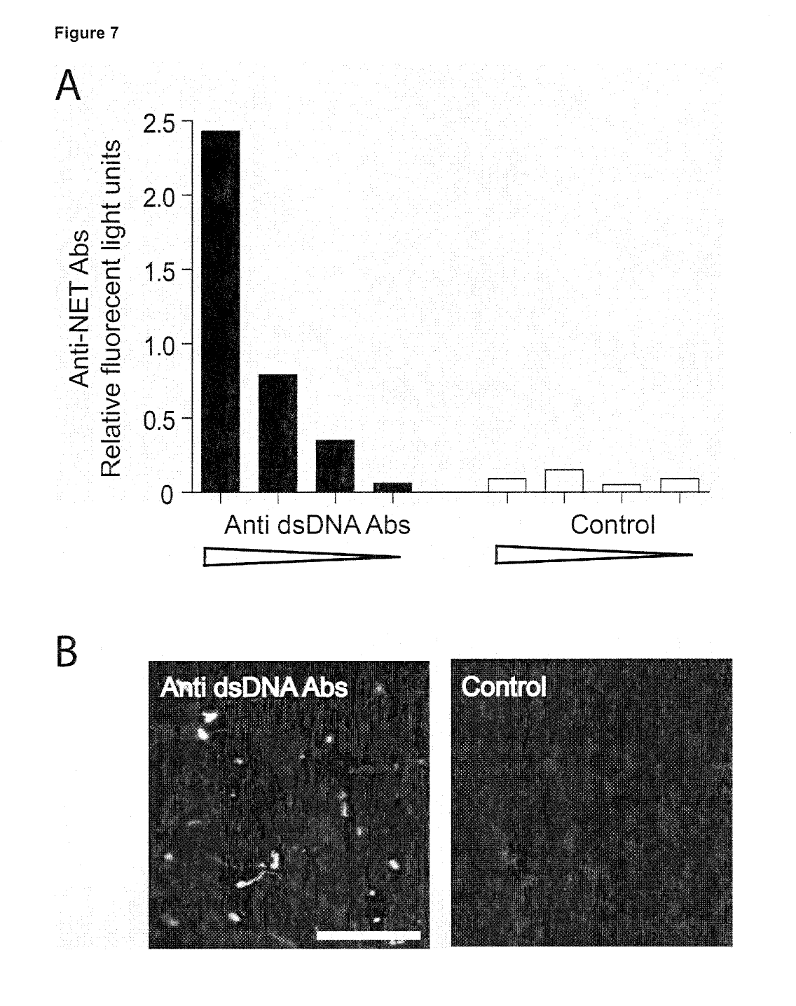

FIG. 7: dsDNA Abs from SLE patient binds to NET

Neutrophils were activated to form NET(s), incubated with different concentrations of an anti-dsDNA antibodies or irrelevant monoclonal antibodies as negative control. The samples were then stained with anti-human secondary antibody (red) and with Sytox® green (green). (A) dsDNA monoclonal antibodies binds to NET(s) in a dose dependent manner when compared to the control antibodies. (B) Representative pictures showing anti-dsDNA monoclonal antibodies binding to NET(s). Scale bars=10 μm.

FIG. 8: NET in the kidney biopsy of a “non-degrader” SLE patient

Projection of a confocal stack of a section of a kidney biopsy of an SLE patient. The sections were stained with antibodies against MPO (green), DNA dye (red) and IgG antibodies (blue). The NET markers and the antibodies localize in the tubuli (Bar=10 μM) and in the glomeruli (Bar=25 μM).

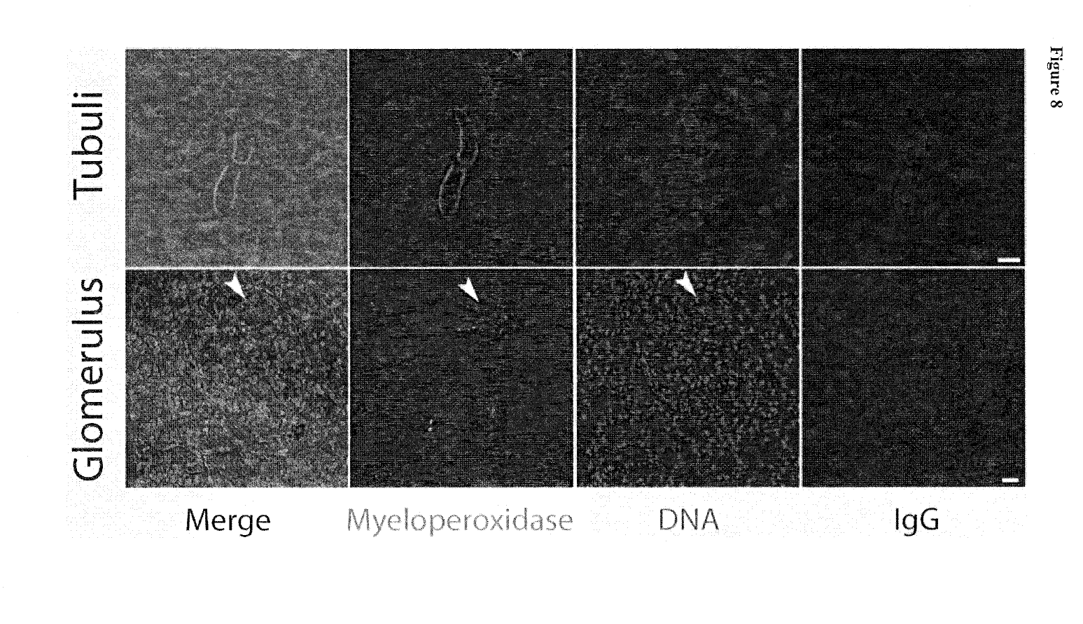

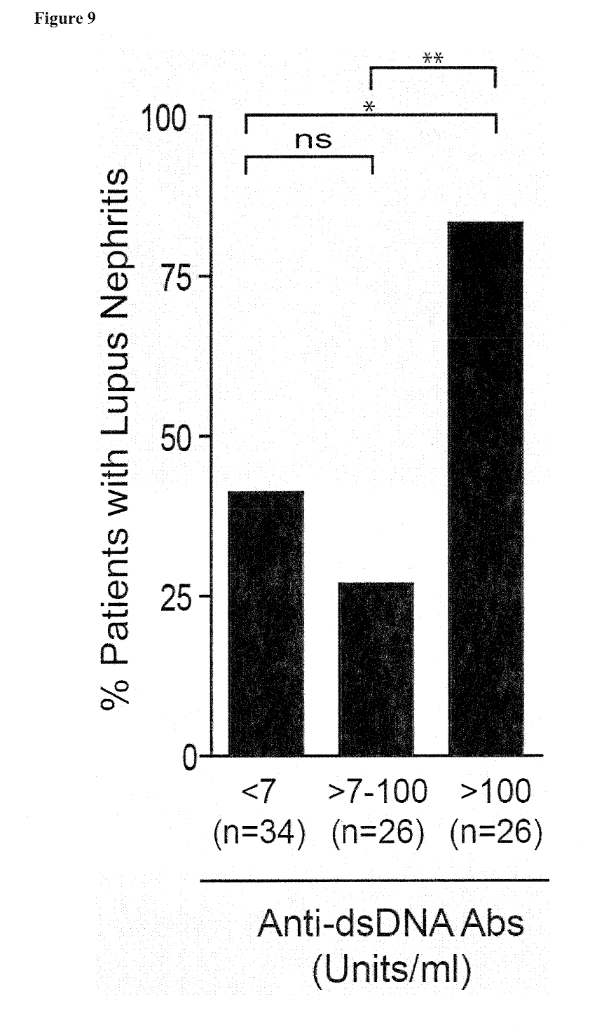

FIG. 9: ds-DNA antibody concentrations correlate with renal disease

Patients with high titers of anti-dsDNA antibodies showed a high incidence of lupus nephritis. The statistics are based on Fisher's exact test. The odds ratios with 95% confidence interval between antibody levels <7 and >7-100 mg ml−1 is 1.900 (0.630-5.729) with ns p value of 0.2866; between >7-100 and >100 mg ml−1 is 13.57 (2.362-77.98) with **p value of 0.0017.

FIG. 10: NET degradation and risk of lupus nephritis

Neutrophils were activated to make NET and incubated with 10% of the indicated sera for 6 h and quantified NET degradation. (A) Healthy control sera degraded NET normally, however, 5 out of 7 SLE patients who were presented with lupus nephritis degraded NET(s) poorly. (B) Table showing the BILAG score of patients sera used for the NET degradation. All the 7 patients had a BILAG score of A.

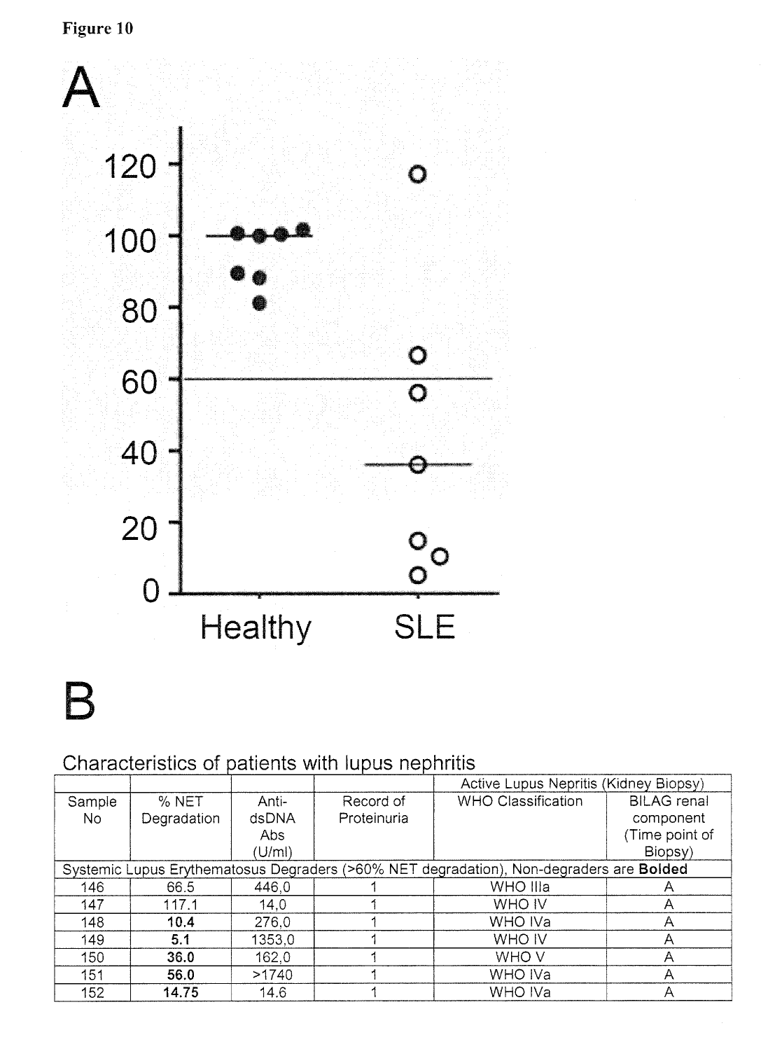

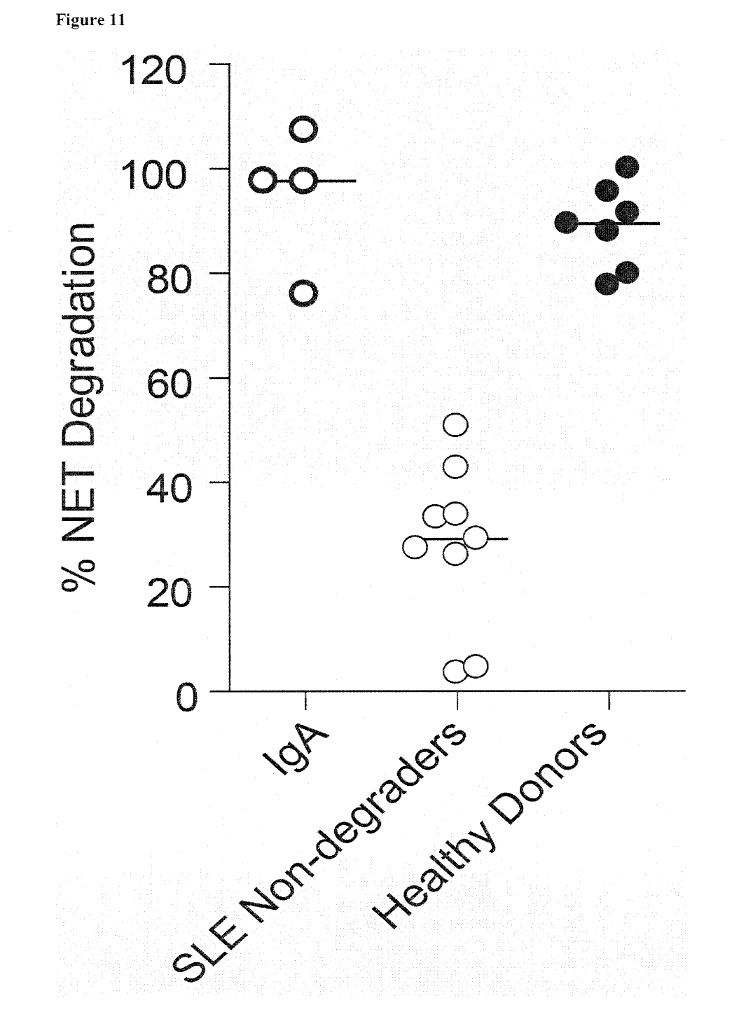

FIG. 11: IgA nephropathy patients' sera degrade NET(s) normally

We activated neutrophils isolated from healthy donors to make NET, incubated them with 10% of the indicated sera for 6 h and quantified NET degradation. IgA nephropathy patients' sera degraded NET as efficiently as healthy donor sera, when compared to SLE non-degrader sera.

FIG. 12: Specificity of NET ELISA

NET were degraded with purified DNase1 and MNase, the latter of which does not degrade NET completely. As controls, cytosol and nucleus of HeLa cells were separates. Neither of them is recognized. See also Example 18.

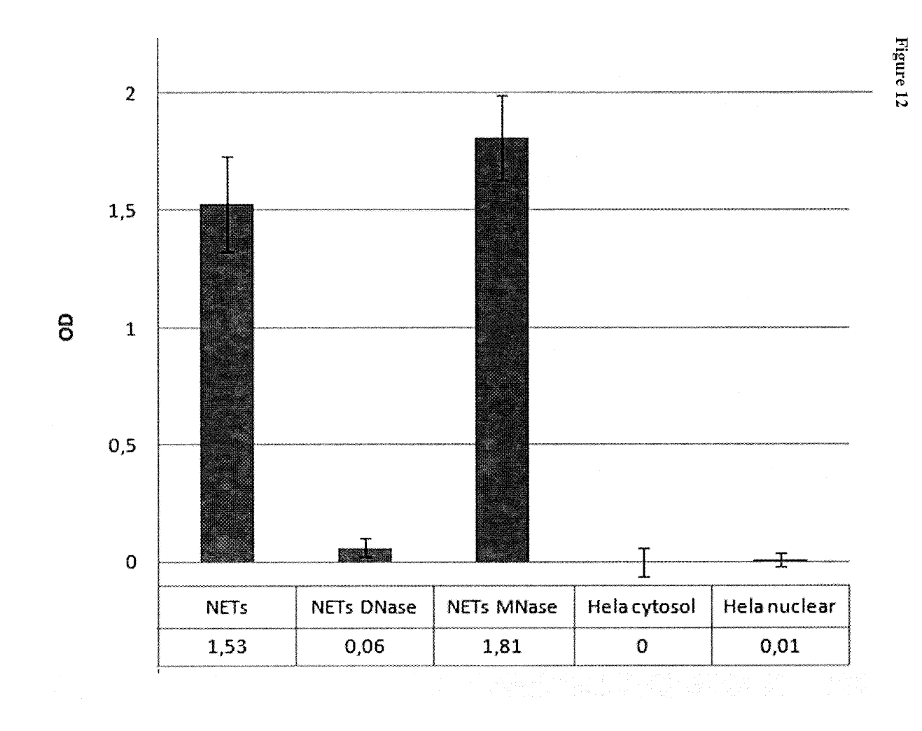

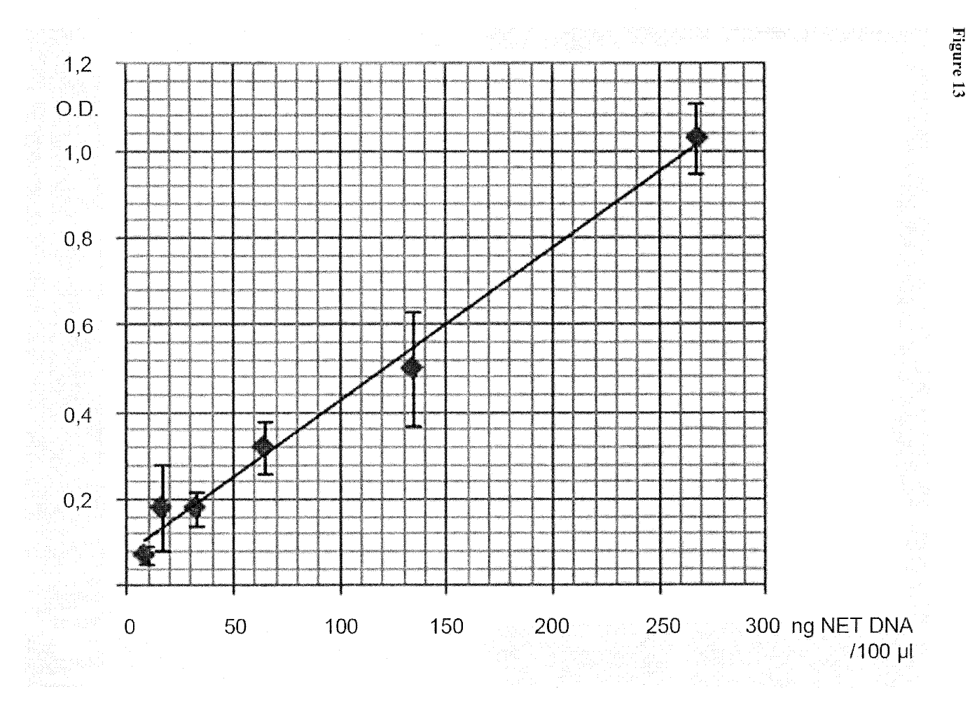

FIG. 13: Sensitivity of ELISA

NET were isolated from neutrophils of healthy donor and assayed in ELISA. The ELISA provided herein can detect concentrations as low as 10 ng NET DNA per 100 μl.

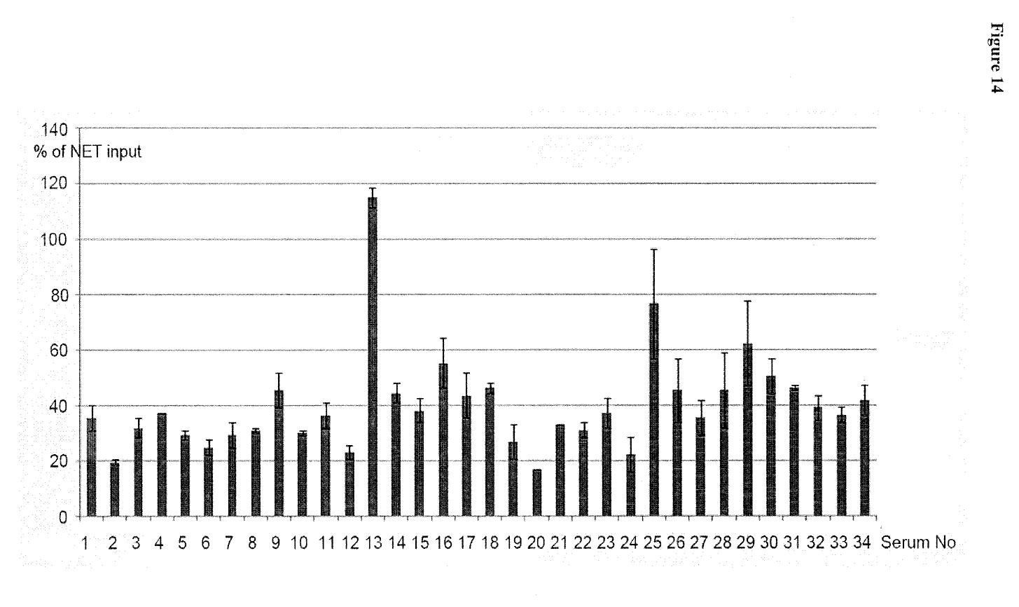

FIG. 14: Reduced degradation of NET by some SLE sera

NET were incubated with serum from SLE patients. Patients nos. 13, 25 and 29 have low NET degradation capability. These patients have a higher risk of developing renal manifestations.

The Examples illustrate the invention.

### Example 1

### Donors, Patients and Clinical Diagnosis

Sixty one unrelated patients with SLE (59 female, 2 male, female:male ratio of 29:1) from the Department of Internal Medicine 3, University Hospital of Erlangen-Nuremberg were randomly selected irrespective of severity or stage of disease. Additionally, 54 healthy unrelated blood donors and 30 patients with rheumatoid arthritis served as control groups. All SLE patients fulfilled the 1982 and 1997 revised criteria of the American College of Rheumatology (ACR) for the diagnosis of SLE (Hochberg, Arthritis Rheum (1997), pp. 1725; Tan et al., Arthritis Rheum (1982), 25:1271-1277). Patients had a median of 13 visits, varying from 1 to 54. Clinical data on disease manifestations of SLE, including proteinuria, nephritic sediments, results of kidney biopsies, arthritis, as well as anti-double stranded (ds) DNA antibody concentrations were retrospectively collected from the patient records. The clinical diagnosis of lupus nephritis was based on histological examination of kidney biopsies, nephritic urine sediments and proteinuria. All patients with proven lupus nephritis, even if resolved, were considered as having renal manifestation of SLE. In addition, 7 patients that already had nephritis and a BILAG score of A and tested them for NET degradation were selected.

Antibodies against dsDNA in human serum were quantified using an in vitro diagnostic radio immunoassay (IBL, Hamburg). Antibody titers against nuclear components in human serum were quantified using indirect immunofluorescence on fixed Hep2 cells. Proteinuria and hematuria were semi-quantitatively assessed using reagent strips for urine analysis. Urine sediments were analysed by light microscopy. Proteinuria was quantified in urine collected over 24 h.

### Example 2

### Neutrophils and Sera

Human neutrophils were isolated from blood obtained from the blood bank in a protocol approved by the ethics committee of the Charité Hospital, Berlin. We isolated neutrophils by density gradient separation (Aga et al., J Immunol (2002), 169:898-905). The cells were seeded onto tissue culture plates or on cover-slips and activated with phorbol myristate acetate (PMA) for NET formation. Serum was obtained from venous blood and aliquoted and stored at −20° C. until use. For antibody depletion, sera were incubated with a protein A/G Ultra-Link Resin (Thermo Scientific) according to the manufacturer's instructions.

### Example 3

### Isolation of Neutrophils and Induction of NET Formation

### Isolation of Neutrophils:

Neutrophils were isolated using the Percoll® gradient method. Peripheral venous blood from a healthy donor was drawn using heparin (Ratiopharm) at a concentration of 10 U/ml. Then 5 ml of Histopaque-1119® (Sigma) were layered in 15 ml falcon tube using a sterile pipette. The number of falcon tubes is calculated based on the amount of blood that is used for neutrophil isolation. Herein, the method has been employed for the amount of cells required for one 96 well plate. 5 ml of blood from the tubes were carefully layered on the Histopaque-1119®, using Pasteur pipette. Then the tubes were centrifuged for 20 min at 800 g at room temperature. Thereby, blood was separated into plasma (top phase), PBMCs (interphase), neutrophils contaminated with red blood cells (RBCs; bottom phase) and RBCs (pellet). The top phase was carefully discarded using a vacuum pump with a sterile glass Pasteur pipette. The red diffused layer or neutrophil rich bottom phase was collected and transferred to a fresh 15 ml sterile falcon tube using Pasteur pipette. The RBC pellet was discarded. One volume was then mixed with three volumes of washing buffer consisting of PBS (Invitrogen) supplemented with 0.5% HSA (Grifols). The tubes were closed tightly and flipped 3-5 times until a homogenous colour could be seen. Then the tubes were centrifuged at 300 g at room temp for 10 min. Subsequently, a red pellet in the falcon tubes could be seen. The supernatants were carefully discarded using a vacuum pump with a sterile glass Pasteur pipette. The cell pellet was then resuspended in 2-3 ml of washing buffer. Meanwhile, the gradient was prepared with sterile Percoll® (GE Healthcare Life Sciences). This is a discontinuous gradient consisting of Percoll®-layers with densities of 1.105 g/ml, 1.100 g/ml, 1.093 g/ml, 1.087 g/ml, and 1.081 g/ml.

### Gradient Preparation:

36 ml of sterile Percoll® and add 4 ml of 10×PBS was added to form the gradient stock. Following this, for the preparation of gradients of 85, 80, 75, 70 and 65%, gradient stock and PBS were added as follows to falcons marked with the representative gradients:

| Conc. Of Gradient | Gradient Stock | 1 x PBS |
|---|---|---|
| 85% | 8.5 ml | 1.5 ml |
| 80% | 8.0 ml | 2.0 ml |
| 75% | 7.5 ml | 2.5 ml |
| 70% | 7.0 ml | 3 ml |
| 65% | 6.5 ml | 3.5 ml |

Using sterile Pasteur pipette, 2 ml of 85% gradient was added in a 15 ml falcon tube. Following that, 2 ml of 80% gradient was carefully layered. This was followed in sequential order until a 65% gradient. At the end of this, the falcon contained gradients ranging from 85% to 65% with the final volume of 10 ml. Subsequently, the resuspended cells from step 11 were carefully layered onto the gradient prepared previously. Then the tubes were centrifuged for 20 min at 800 g at room temperature. The interphase between 70-75% Percoll® layers was collected and transferred to a fresh falcon tube. This layer looks like a white cloud. Following this, the falcon tube was filled with the layer with PBS-HSA. The tubes were closed tightly and flipped 3-5 times until they were homogenous. Then the tubes were centrifuged at 200 g at room temp for 10 min. The supernatant was discarded and the pellet resuspended with 2 ml of RPMI-Hepes medium.

### Cell Counting:

10 μl of the resuspended cells was transferred into 90 μl of tryphan blue and mixed it well with the pipette. A hemocytometer was used and 10 μl of the mixed cells and tryphan blue were added onto the slides. The cells were counted with a cell counter under the 10× objective in a light microscope. Finally, the cells were diluted in medium to the required concentration and kept at room temperature until further use.

### Culture Conditions:

Neutrophils (1×10⁶/ml) were suspended in RPMI medium (phenol red-free) supplemented with 10 mM Hepes and 50 μl were seeded into 96 well tissue culture plates.

### Induction of NET Formation:

The cells were activated with phorbol myristate acetate (PMA, 20 nM) (Sigma) and incubated at 37° C. and 5% CO₂ for 5 h for formation of NET(s). Micrococcal nuclease (1 U/ml final concentration) was added and incubated for 10 min at 37° C. The supernatant was removed and filled into 15 ml jars. The mixture was allowed to sediment for 10 min at 20 g. The supernatant contained NET.

### Example 4

### Bioassay for NET Degradation

Wells containing NET(s) were incubated with 1 U/ml MNase or DNase1 (both from Worthington) (for 100% degradation control) for 10 min or 10% human serum for 6 h (patient body fluid sample and control body fluid sample). 2 mM EDTA was added to stop nuclease activity and the culture supernatants were collected. 2 μM of Picogreen (Invitrogen), a DNA fluorescent DNA dye, was added and quantified DNA by fluorescence spectrometry (Fuchs et al., J Cell Biol (2007), 176:231-241). The amount of NET-DNA released with MNase or DNase1 was considered as “100% NET degradation”. For analysis of the cohort's sera, NET degradation by the serum of the healthy neutrophil donor (control sample) was considered to be 100%. In some cases, NET(s) were treated with 10% sera spiked with 1 U/ml DNase1 or MNase before quantification of NET degradation.

### Example 5

### Immunfluorescence Microscopy

NET(s) were fixed with 2% PFA (Merck 4005) and washed 3 times for 5 min each. Fixed NET(s) were incubated with primary antibodies to the anti-H2A-H2B-DNA complex (Losman et al., J Immunol (1992), 148: 1561-1569), with anti-myeloperoxidase antibodies (Dako, A0398), or human sera, and bound antibodies were detected with respective secondary antibodies (donkey anti-mouse; donkey anti-rabbit; donkey anti-human) coupled to Cy2 or Cy3 (Jackson Immuno). DNA was stained with Draq5 (Biostatus). Isotype-matched controls were used. The specimens were processed and analysed as described previously (Fuchs et al., J Cell Biol (2007), 176:231-241).

### Example 6

### Cell Based NET-Assay

Neutrophils were activated to allow maximum NET formation and then washed and fixed. The NET(s) were incubated with 1/100 dilution of serum for 1 h at 37° C. The secondary antibody, anti-human IgG coupled with Cy3 (Jackson), was added and incubated for 1 h at room temperature. Thereafter, the NET(s) were stained with 2 μM Sytox Green®. The fluorescence was measured with two channels 518/590 nm and 485/518 nm, respectively. The Cy3 signal value was normalised to the Sytox signal, so the value was proportional to the amount of NET(s) in each well. The results were plotted as relative fluorescence light units. There were elevated levels of anti-NET antibodies in non-degraders. This indicates that inefficient NET degradation might be linked to high titers of anti-NET antibodies in vivo. This retrospective quantification of anti-NET antibodies using the cell based NET-Assay (as described above) in the sera could be used as a diagnostic tool to predict the levels of anti-NET antibodies that might prevent NET degradation. SLE sera, in particular those from non-degraders, contained high levels of anti-NET antibodies (FIG. 4A). Furthermore, consistent with the model that anti-NET antibodies protect NET(s) from DNase1 degradation, these antibodies were particularly abundant in Group 2. This was confirmed by microscopy: sera from non-degraders, both from Group 1 and 2 bound to NET(s) at the tested concentration (representative micrographs shown in FIG. 6). As controls, sera from healthy donors or SLE degraders did not recognize NET(s) either by ELISA or by immunofluorescence. These data indicate that inefficient NET degradation correlates with high levels of anti-NET antibodies.

### Example 7

### Statistical Analyses

The normality of the data was checked by a Shapiro-Wilk normality test (Shapiro et al., Biometrika (1965), 52: 591-611). For unpaired comparisons of two groups, a t-test (David et al., The American Statistician (1997), 51: 9-12) and, in the case of non-normality, a nonparametric Wilcoxon test (Wilcoxon, Biometrics (1945), 1: 80-83) was performed. For more than two unpaired groups, a variance analytical approach was employed and comparison was done with an ANOVA and Kruskal-Wallis test (Kruskal, J Am Stat Ass (1952), 47: 583-621) in case of normality. In order to adjust the alpha level, Dunnett's post-hoc tests (Dunnett et al., J Am Stat Ass (1995), 50: 1096-1121) were used. Paired comparisons of two groups were performed with a paired t-test or with a paired Wilcoxon test in the case of non-normality. An ANOVA with repeated measurements or a Friedman test (Friedmann et al., Am Stat Ass (1937), 32: 675-701) were performed for a paired comparison of more than two groups and Dunn's post test (Dunn, Technometrics (1964), 5: 241-252) was used. For clinical data analysis, the 95% confidence interval and the odds ratio were calculated. The overall level of significance was set at p<0.05.

### Example 8

### Quantification of NET Degradation

### Degradation of NET:

After induction of NET formation as described in Example 3, the supernatants from the wells were carefully discarded with a multi-well pipette. Then, 500 of fresh medium was added to all wells. Following this, 3 wells (triplicates) in the plate were marked as “STANDARD” and 6 wells (3×2) as “CONTROL 1” and “CONTROL 2”, respectively. The respective plates contained the following: STANDARD: MNase (degradation control); CONTROL 1: Normal serum of healthy donors (control sample); and CONTROL 2: Medium alone. The “STANDARD” plate containing only MNase was used as control for degradability of the NET(s). For the calculation, CONTROL 1 (control sample of healthy donor) was compared to TEST (patient sample of an SLE patient). Based on the number of samples to be tested, multiplied by three, plates were labelled as “TEST” (patient sample). For both, the control and the patient samples, the serum was diluted to 10% serum final concentration (The total volume of the assay is 100 μl, since 50 μl of medium was added to the NET(s) previously and 10 μl of total serum was added to 40 μl of medium). This was the patient sample. The 50 μl of the CONTROL 1 (healthy donor; control sample) and the TEST (patient sample) serum to the respective labelled wells were added and incubated at 37° C. and 5% CO₂ for 6 h. Furthermore, 50 μl of medium were added to CONTROL 2 (medium alone). After the incubation, the plates were taken out and 50 μl of 1 U/ml micrococcal nuclease (MNase; Worthington Biochemical Corp.) were added to the wells labelled as “STANDARD” and incubated for 10 min at 37° C. Subsequently, 2 mM of EDTA (2 μl volume) was added to all wells to stop nuclease activity. Then, the supernatants were carefully collected and transferred to a new round bottom 96 well plate.

### Quantification of NET Degradation Using Fluorometer:

For the quantification, 34 μl of the supernatant were transferred to a Nunc Black 96 well plate. Then, 70 μl of Picogreen (Invitrogen) diluted with 1×TE buffer according to manufacturer's instructions was added. The plate was read in the fluorometer (Fluoroscan) at 485/518 nm. For calculation, the value of the TEST, STANDARD and CONTROL 1 was subtracted with the value of the CONTROL 2 (Medium). The NET degradation level of CONTROL 1 (control sample of healthy donor) was set as 100% degradation. To calculate the percent NET degradation of a patient sample compared to the NET degradation of the control sample, the following formula was used:

% NET degradation=(NET degradation level of patient sample/NET degradation level of control sample)×100

### Example 9

### Serum DNase1 Degrades NET(s)

To analyze how NET(s) are degraded, it was first analysed if there are neutrophil factor(s) that degrade NET(s). In vitro, they were stable for more than 90 hours in the absence of serum (FIG. 1A) (at each time point, the wells were treated with MNase for 10 min to digest NET(s)), although they can experimentally be digested with nucleases such as micrococcal nuclease (MNase) (Fuchs, J Cell Biol (2007), 176:231-241). However, NET(s) were degraded after incubation with 10% serum isolated from healthy donors (FIG. 1B). Sera of 11 different healthy donors were initially tested for NET degradation and did not exhibit significant variation (FIG. 2). These data were confirmed by microscopy. The formation of NET(s) was induced as described above, incubated with sera and stained the NET(s) were later with 5 μM DNA dye Draq5 (Biostatus) and antibodies (5 μg/ml) that recognize myeloperoxidase (anti-MPO rabbit polyclonal antibody, Dako, A0398) and Histone 2A/H2B/DNA (Losman et al., J Immunol (1992), 148:1561-1569) (FIG. 1D). NET degradation was dose- and time dependent (FIGS. 1B-C), suggesting an enzymatic activity.

To identify the serum factor(s) responsible for NET disassembly, the requirement of divalent cations was investigated, a cofactor of several nucleases. EGTA, a calcium-specific chelator, prevented NET degradation. This inhibition was reverted with addition of exogenous calcium (FIG. 1E). The calcium dependence of NET degradation suggests that serum DNase 1 degraded the NET(s). This extracellular, neutral endonuclease is mainly produced in the pancreas and secreted into the digestive system and the blood stream (Suck et al., Nature (1988), 332:464-468; Liu et al., Aim N Y Acad Sci (1999), 887: 60-76). For reasons that are not completely understood, G-actin forms a complex and inhibits DNase1 (Kabsch et al., Nature (1990), 347:37-44). The biological significance of this association is not understood (Lazarides et al., Proc Natl Acad Sci USA (1974), 71:4742-4746). This property of G-actin was used to show that it inhibited NET degradation in a dose dependent manner (FIG. 1F), indicating that DNase1 degrades NET(s). Anti-DNase1 antibodies, but not an irrelevant antibody control against Neutrophil Elastase, prevented serum-mediated NET degradation (FIG. 1H) confirming that DNase 1 digests NET(s). In addition, as controls, it was shown that inhibitory concentrations of either G-actin (FIG. 1G) or anti-DNase1 antibodies (FIG. 1I) block DNase1, but not MNase-mediated NET disintegration. These data indicate that serum DNase1 is essential for NET degradation. Degradation of NET(s) is a novel function for DNase1. In the present invention, it is indicated that the dysfunction of this enzyme could be linked to immunopathogenesis of SLE. Hence, it is assumed that insufficient NET degradation by DNase1 would allow NET(s) to persist and, thus, to foster the presentation of self-antigens, a process which may promote SLE.

### Example 10

### Impaired NET Degradation in a Subset of SLE Patients

The NET degradation activity of 145 sera collected from: (i) 54 healthy unrelated blood donors (ii) 30 rheumatoid arthritis (RA) patients and (iii) 61 unrelated patients with documented SLE (59 female, 2 male, female:male ratio of 29:1) was analyzed at different disease stages and activities (Table 2). NET degradation activity of these sera was tested in a double blind experiment on NET(s) produced by neutrophils isolated from a healthy donor. The NET degradation activity of the sera from this donor was defined as 100%. 60% of NET degradation was set as cut-off and patient sample donors with values higher than 60% were termed “degraders”. Those with less than 60% NET degradation were termed “non-degraders”. In the NET degradation assay, sera from healthy donors degraded NET(s) efficiently, with a mean percent degradation of 98.1% and a standard deviation (SD) of 8.2 (FIG. 3A). All but one of the sera from control RA patients degraded NET(s) normally (mean 91.3%±16.7). Interestingly, two populations were identified in the sera of SLE patients: 63.9% were “degraders” and 36.1% were “non-degraders”. These results were confirmed by microscopy. Accordingly, it was observed that NET(s) were degraded by sera from healthy donors (FIG. 3B) and by sera from a subgroup of SLE patients (FIG. 3C). It was also corroborated that the sera of a subgroup of SLE patients was unable to degrade NET(s) (FIG. 3D). Thus, a subpopulation of SLE patients displayed poor NET degradation in vitro.

TABLE 2
Characteristics of Patients and Control Sera included in the Cohort

| | | | Anti- | | | |
|---|---|---|---|---|---|---|
| | Co- | % NET | dsDNA | | Anti-Nuclear | |
| S. | hort | Degrada- | anti- | Pro- | antibodies | |
| No | No. | tion | bodies | teinuria | Pattern | Titer |

Healthy Donor

| | | | | | | |
|---|---|---|---|---|---|---|
| 1 | 9 | 93.1 | — | — | — | — |
| 2 | 25 | 97.2 | — | — | — | — |
| 3 | 27 | 94.8 | — | — | — | — |
| 4 | 28 | 94.7 | — | — | — | — |
| 5 | 29 | 102.5 | — | — | — | — |
| 6 | 30 | 78.2 | — | — | — | — |
| 7 | 31 | 93.8 | — | — | — | — |
| 8 | 80 | 99.7 | — | — | — | — |
| 9 | 81 | 100.1 | — | — | — | — |
| 10 | 82 | 99.2 | — | — | — | — |
| 11 | 83 | 96.9 | — | — | — | — |
| 12 | 84 | 96.9 | — | — | — | — |
| 13 | 85 | 98.5 | — | — | — | — |
| 14 | 95 | 96.1 | — | — | — | — |
| 15 | 96 | 83.0 | — | — | — | — |
| 16 | 97 | 96.2 | — | — | — | — |
| 17 | 101 | 94.2 | — | — | — | — |
| 18 | 102 | 87.9 | — | — | — | — |
| 19 | 103 | 97.8 | — | — | — | — |
| 20 | 104 | 90.2 | — | — | — | — |
| 21 | 105 | 92.7 | — | — | — | — |
| 22 | 106 | 97.5 | — | — | — | — |
| 23 | 107 | 92.2 | — | — | — | — |
| 24 | 108 | 93.8 | — | — | — | — |
| 25 | 176 | 97.9 | — | — | — | — |
| 26 | 177 | 99.4 | — | — | — | — |
| 27 | 178 | 106.8 | — | — | — | — |
| 28 | 179 | 101.2 | — | — | — | — |
| 29 | 180 | 93.5 | — | — | — | — |
| 30 | 181 | 86.7 | — | — | — | — |
| 31 | 182 | 101.6 | — | — | — | — |
| 32 | 183 | 89.2 | — | — | — | — |
| 33 | 184 | 101.2 | — | — | — | — |
| 34 | 185 | 96.5 | — | — | — | — |
| 35 | 186 | 106.2 | — | — | — | — |
| 36 | 187 | 108.7 | — | — | — | — |
| 37 | 188 | 75.2 | — | — | — | — |
| 38 | 189 | 95.4 | — | — | — | — |
| 39 | 190 | 102.7 | — | — | — | — |
| 40 | 191 | 105.0 | — | — | — | — |
| 41 | 192 | 104.0 | — | — | — | — |
| 42 | 193 | 111.0 | — | — | — | — |
| 43 | 194 | 121.4 | — | — | — | — |
| 44 | 195 | 101.3 | — | — | — | — |
| 45 | 219 | 111.8 | — | — | — | — |
| 46 | 220 | 112.2 | — | — | — | — |
| 47 | 221 | 111.5 | — | — | — | — |
| 48 | 222 | 96.1 | — | — | — | — |
| 49 | 223 | 99.3 | — | — | — | — |
| 50 | 225 | 95.0 | — | — | — | — |
| 51 | 255 | 91.6 | — | — | — | — |
| 52 | 256 | 100.7 | — | — | — | — |
| 53 | 257 | 99.9 | — | — | — | — |
| 54 | 258 | 107.6 | — | — | — | — |

RheumatoidArthritis

| | | | | | | |
|---|---|---|---|---|---|---|
| 55 | 5 | 88.4 | — | — | — | — |
| 56 | 6 | 76.3 | — | — | — | — |
| 57 | 8 | 70.4 | — | — | — | — |
| 58 | 11 | 63.2 | — | — | — | — |
| 59 | 12 | 74.8 | — | — | — | — |
| 60 | 17 | 88.2 | — | — | — | — |
| 61 | 18 | 97.5 | — | — | — | — |
| 62 | 198 | 76.7 | — | — | — | — |
| 63 | 199 | 68.9 | — | — | — | — |
| 64 | 200 | 100.2 | — | — | — | — |
| 65 | 201 | 99.6 | — | — | — | — |
| 66 | 202 | 108.2 | — | — | — | — |
| 67 | 203 | 95.6 | — | — | — | — |
| 68 | 204 | 92.8 | — | — | — | — |
| 69 | 205 | 109.1 | — | — | — | — |
| 70 | 207 | 114.1 | — | — | — | — |
| 71 | 208 | 79.2 | — | — | — | — |
| 72 | 209 | 106.0 | — | — | — | — |
| 73 | 210 | 114.4 | — | — | — | — |
| 74 | 211 | 109.9 | — | — | — | — |
| 75 | 212 | 105.4 | — | — | — | — |
| 76 | 213 | 96.7 | — | — | — | — |
| 77 | 214 | 101.2 | — | — | — | — |
| 78 | 215 | 101.4 | — | — | — | — |
| 79 | 216 | 104.4 | — | — | — | — |
| 80 | 217 | 90.8 | — | — | — | — |
| 81 | 218 | 108.6 | — | — | — | — |
| 82 | 230 | 64.6 | — | — | — | — |
| 83 | 250 | 57.7 | — | — | — | — |
| 84 | 90 | 73.4 | — | — | — | — |

Systemic Lupus Erythematosus
Degraders (>60% NET degradation)

| | | | | | | |
|---|---|---|---|---|---|---|
| 85 | 32 | 92.7 | 0.1 | −1 | granular | 1:100 |
| 86 | 35 | 69.9 | 92 | −1 | homo | 1:3200 |
| 87 | 37 | 91.4 | 12.8 | −1 | homo | 1:320 |
| 88 | 38 | 94.8 | 0.1 | −1 | granular | 1:1000 |
| 89 | 39 | 64.0 | 22.1 | −1 | homo | 1:1000 |
| 90 | 40 | 82.5 | 0.1 | 1 | homo | 1:1000 |
| 91 | 41 | 88.4 | 13.8 | 1 | homo | 1:3200 |
| 92 | 44 | 89.5 | 0.1 | −1 | centromer, | 1:3200 |
| | | | | | homo | |
| 93 | 46 | 97.6 | 0.1 | −1 | nuclear | 1:3200 |
| 94 | 47 | 97.4 | 8.8 | −1 | nuleolar | 1:320 |
| 95 | 48 | 98.3 | 6.5 | 1 | homo | 1:320 |
| 96 | 51 | 88.0 | 33.9 | −1 | homo | 1:10000 |
| 97 | 52 | 99.0 | 0.1 | −1 | granular/ | 1:320 |
| | | | | | nucleolar | |
| 98 | 54 | 91.5 | 6 | −1 | granular | 1:1000 |
| 99 | 55 | 93.2 | 12.3 | −1 | homo | 1:1000 |
| 100 | 56 | 94.6 | 4.8 | 1 | homo | 1:320 |
| 101 | 58* | 93.8 | 0.1 | −1 | — | negative |
| 102 | 59 | 94.9 | 0.1 | −1 | homo | 1:100 |
| 103 | 60 | 80.4 | 0.1 | 1 | granular | 1:3200 |
| 104 | 61 | 81.3 | 18.3 | −1 | homo/ | 1:1000 |
| | | | | | nucleolar | |
| 105 | 63 | 83.7 | 13.5 | −1 | homo | 1:3200 |
| 106 | 64 | 85.6 | 39 | −1 | homo | 1:1000 |
| 107 | 66 | 77.9 | 5.3 | −1 | homo | 1:1000 |
| 108 | 67 | 81.6 | 0.1 | −1 | granular/ | 1:1000 |
| | | | | | nucleolar | |
| 109 | 69 | 95.7 | 8.5 | −1 | homo | 1:1000 |
| 110 | 73 | 95.2 | 0.1 | −1 | — | negative |
| 111 | 76 | 82.8 | 0.1 | 1 | granular | 1:3200 |
| 112 | 87 | 88.9 | 15.7 | 1 | granular | 1:3200 |
| 113 | 88 | 78.9 | 74.1 | 1 | granular | 1:100 |
| 114 | 224 | 91.1 | 13.7 | −1 | — | negative |
| 115 | 226 | 76.7 | 0.1 | −1 | homo | 1:320 |
| 116 | 232 | 68.2 | 10.3 | 1 | homo | 1:10000 |
| 117 | 233 | 77.2 | 4.2 | 1 | granular | 1:100 |
| 118 | 236 | 95.6 | 0.1 | −1 | granular | 1:100 |
| 119 | 238 | 81.1 | 19.8 | −1 | homo | 1:1000 |
| 120 | 239 | 74.3 | 3.5 | −1 | granular | 1:320 |
| 121 | 243 | 90.8 | 4.2 | −1 | homo/ | 1:3200 |
| | | | | | nucleolar | |
| 122 | 244 | 76.4 | 0.1 | 1 | granular | 1:1000 |
| 123 | 253 | 77.5 | 0.1 | −1 | homo | 1:320 |

Non-Degraders (<60% NET degradation)
Group 1 SLE sera

| | | | | | | |
|---|---|---|---|---|---|---|
| 124 | 2 | 12.3 | 23.4 | 1 | — | negative |
| 125 | 34 | 21.3 | 140 | −1 | homo | 1:100 |
| 126 | 62 | 49.9 | 0.1 | 1 | homo/ | 1:320 |
| | | | | | granular | |
| 127 | 65 | 3.6 | 314 | 1 | homo | 1:1000 |
| 128 | 71 | 30.4 | 21.9 | −1 | homo | 1:3200 |
| 129 | 94* | 33.0 | 51.6 | 1 | granular/ | 1:1000 |
| | | | | | homo | |
| 130 | 197 | 29.3 | 36.4 | −1 | homo | 1:3200 |
| 131 | 229 | 16.0 | 39.1 | 1 | homo | 1:10000 |
| 132 | 240 | 53.9 | 136 | 1 | homo | 1:10000 |
| 133 | 242 | 37.6 | 0.1 | 1 | granular | 1:1000 |
| 134 | 248 | 59.6 | 0.1 | 1 | granular | 1:10000 |
| 135 | 251 | 54.7 | 1130 | 1 | homo | 1:3200 |
| 136 | 254 | 12.6 | 43.4 | −1 | homo | 1:3200 |

Group 2 SLE sera

| | | | | | | |
|---|---|---|---|---|---|---|
| 137 | 36 | 0.9 | 690 | 1 | homo | 1:3200 |
| 138 | 50 | 55.6 | 290 | 1 | granular | 1:10000 |
| 139 | 72 | 1.2 | 1950 | 1 | homo/ | 1:3200 |
| | | | | | granular | |
| 140 | 75 | 0.1 | 7.2 | −1 | homo | 1:3200 |
| 141 | 89 | 20.1 | 1782 | −1 | granular | 1:3200 |
| 142 | 206 | 3.1 | 1960 | 1 | granular | 1:1000 |
| 143 | 237 | 21.3 | 2803 | 1 | homo | 1:3200 |
| 144 | 247 | 10.5 | 2192 | 1 | homo | 1:3200 |
| 145 | 249 | 10.0 | 1130 | 1 | homo/ | 1:3200 |
| | | | | | granular | |

Numbers in “Bold” are non-degraders
— Not applicable or Not available
1 Proteinuria positive (Grey Shaded)
−1 Proteinuria negative
Homo—Homogenous

### Example 11

### Inhibitory Mechanisms of NET-Degradation

To elucidate why some SLE sera cannot digest NET(s), DNase1 was exogenously added to the sera. Two possible results were anticipated: (1) “Spiking” with exogenous DNase1 would restore the NET degradation, implying a non-functional DNase 1. (2) “Spiking” would not restore the serum activity, suggesting the presence of either DNase1 inhibitors or the physical protection of NET(s) from this enzyme. To further clarify the second option, sera was also “spiked” with MNase. Digestion of NET(s) which were not digested by DNase1 with MNase would indicate the presence of DNase 1-specific inhibitors (“Group 1”). In contrast, if MNase would not restore NET degradation activity, this would indicate the general “protection” of NET(s) from nucleases (“Group 2”).

Addition of DNase1 to “non-degrader” SLE sera did not restore NET degradation to the level of controls (FIG. 4A). These data suggested that mutations in DNase1 did not account for impaired NET degradation in the cohort used herein (Yasutomo et al., Nat Genet (2001), 28:313-314; Bodano et al., Arthritis Rheum (2004), 50:4070-4071). Spiking these sera with MNase identified two groups of patient's sera. In Group 1, addition of MNase restored NET degradation (from median 29.7% to 95.2%), indicating the presence of DNase1-specific inhibitor(s) (FIG. 4A). In Group 2, addition of MNase resulted only in a marginal increase in NET degradation from median 10.5% to 20.9%, indicating the presence of factor(s) that protect NET(s) from enzymatic degradation.

### Example 12

### NET-Protecting Antibodies in SLE Sera Prevent DNase1 Degradation of NET(s)

It was tested whether the sera in Group 2 contained NET “protecting” antibodies that block the access of nucleases to NET(s). To analyze this, these sera were depleted of antibodies using protein A/G beads. FIG. 4B shows that sera in Group 2 efficiently digested NET(s) after antibody depletion (median 19.9% before and 78% after antibody depletion,). In contrast, NET degradation increased only slightly in Group 1 sera (median 29% before and 43% after antibody depletion). These data indicate that sera of Group 2 contain antibodies that shield the NET(s) from nucleases. Taken together, these data show that NET degradation is prevented either by inhibiting DNase1 (Group 1) or by covering NET(s) with antibodies and protecting them from endonuclease digestion (Group 2).

### Example 13

### Elevated Levels of Anti-NET Antibodies in Non-Degraders

It was proposed that inefficient NET degradation might be linked to high titers of anti-NET antibodies in vivo. To test this, anti-NET antibodies were retrospectively quantified using a NET-Assay (as described above) in the sera. SLE sera, in particular those from “non-degraders”, contained high levels of anti-NET antibodies (FIG. 5A). Furthermore, consistent with the model that anti-NET antibodies protects NET(s) from DNase1 degradation, these antibodies were particularly abundant in Group 2. This was confirmed by microscopy. Sera from “non-degraders”, both from Group 1 and 2, bound to NET(s) at the tested concentration (representative micrographs shown in FIG. 6). As controls, sera from healthy donors or SLE “degraders” did not recognize NET(s), either by NET Assay or by immunofluorescence (FIG. 5A and FIG. 6). These data indicate that inefficient NET degradation correlates with high levels of anti-NET antibodies.

### Example 14

### Impaired NET Degradation Correlates with Lupus Nephritis

Anti-double stranded (ds) DNA antibodies and anti-nuclear antibodies (ANA) are hallmark tests for SLE diagnosis. Anti-dsDNA antibodies correlate with renal disease and increasing titres may indicate disease flares (Hahn, N Engl J Med (1998), 338:1359-1368). Anti-dsDNA antibody titres and ANA titres were determined at the same clinical visit when the serum samples for the NET degradation assays were taken. FIG. 5B and FIG. 5C show that “non-degraders” have significantly higher anti-dsDNA antibody titres and ANA titers than “degraders”. Sera in Group 2 have higher antibody titres than sera in Group 1, consistent with their NET-protection function. Consistently, it was shown that an anti-dsDNA monoclonal antibody derived from a SLE patient (Winkler et al., Clin Exp Immunol (1991), 85:379-385) binds to NET(s) (FIG. 7). Interestingly, sera of Group 2 have higher antibody titers than those of Group 1, consistent with their NET-protecting function. A frequent and serious manifestation of SLE is glomerulonephritis—a condition that can cause proteinuria and progress to kidney failure (Weening et al., Am Soc Nephrol (2004), 15: 241-250.). A retrospective correlation analysis showed that patients who do not degrade NET(s) developed lupus nephritis significantly more frequently than “degraders” (FIG. 5D). Notably, all “non-degrader” patients, regardless of belonging to Group 1 or 2, were likely to develop lupus nephritis. These data indicate that impaired NET degradation correlates with renal disease. Indeed, IgG deposition on NET(s) was observed in tubuli and glomeruli in the kidney of an SLE patient who degraded NET(s) poorly (FIG. 8). NET(s) were detected by staining with a DNA dye (Darq5, 5 μg/ml) and an anti-MPO antibody (anti-rabbit MPO antibody, Dako, A0398) and co-localized to antibody deposits. Moreover, in the cohort used herein, impaired NET degradation and high concentrations of anti-dsDNA antibodies, a known risk factor (Hahn, N Engl J Med (1998), 338:1359-1368), were both associated with lupus nephritis (FIG. 5D and FIG. 9). These results suggest that defective NET degradation contributes to renal manifestations in SLE pathogenesis, especially glomerulonephritis. To confirm these observations, 7 patients not included in the original cohort that had biopsy-proven lupus were tested. Five of the seven sera obtained around the time point of kidney biopsies did degrade NET(s), supporting the correlation between lack of NET degradation and lupus nephritis (FIG. 10A). A correlation between certain types of lupus nephritis (WHO classification) with NET degradation (Table 3 and FIG. 10B) was not observed. Interestingly, as a control for other nephritis, it was shown that sera from patients with IgA nephropathy (Cederholm et al., Proc Natl Acad Sci USA (1986), 83: 6151-6155) degraded NET(s) (FIG. 11). This is consistent with the observations that these patients do not make antibodies against NET components or against NET(s). Importantly, these results suggest that defective NET degradation contributes to SLE pathogenesis, especially glomerulonephritis.

TABLE 3
Characteristics of Patients with Lupus Nephritis

| | | | | Active Lupus Nephritis (Kidney Biopsy) | | |
|---|---|---|---|---|---|---|
| | | | | | At date of serum | |
| | | Anti-dsDNA | | | withdrawal | |
| Sample | % NET | antibodies | Record | Before serum | (BILAG renal | After serum |
| No | Degradation | [U/ml] | ofProteinura | withdrawal | component) | withdrawal |

Systemic Lupus ErythematosusDegraders (>60% NET degradation)

| | | | | | | |
|---|---|---|---|---|---|---|
| 90 | 82.5 | 0.1 | 1 | (+) | — | — |
| 91 | 88.4 | 13.8 | 1 | + (WHO IV) | + (B) | + |
| 103 | 80.4 | 0.1 | 1 | + | + (A) | ? |
| 111 | 82.8 | 0.1 | 1 | + (WHO V) | — (D) | — |
| 112 | 88.9 | 15.7 | 1 | — | (+) (C) | + |
| 113 | 78.9 | 74.1 | 1 | + | — (D) | — |
| 116 | 68.2 | 10.3 | 1 | + (WHO IV) | (+) (C) | (+) |
| 117 | 77.2 | 4.2 | 1 | + (WHO II-III) | — (D) | — |
| 122 | 76.4 | 0.1 | 1 | + (WHO II) | + (WHO IV & A) | + |
| 124 | 12.3 | 23.4 | 1 | + (WHO IV) | ++ (WHO IV & A) | ? |
| 127 | 3.6 | 314 | 1 | + (IIa) | (+) (C) | (+) |
| 129 | 33.0 | 51.6 | 1 | ? | + (A) | ? |
| 131 | 16.0 | 39.1 | 1 | + | + (WHO III & A/B) | ? |
| 132 | 53.9 | 136 | 1 | + (WHO IV) | (+) (C) | (+) |
| 133 | 37.6 | 0.1 | 1 | + (WHO V) | + (A) | ? |
| 134 | 59.6 | 0.1 | 1 | + | — (D) | — |
| 135 | 54.7 | 1130 | 1 | + | + (WHO IV) | + |
| 137 | 0.9 | 690 | 1 | + | — (D) | — |
| 138 | 55.6 | 290 | 1 | + | (+) (C) | + |
| 139 | 1.2 | 1950 | 1 | — | — (E) | + |
| 142 | 3.1 | 1960 | 1 | — | + (B) | + |
| 143 | 21.3 | 2803 | 1 | (+) | + (C) | + |
| 144 | 10.5 | 2192 | 1 | + | (+) (C) | ++ |
| 145 | 10.0 | 1130 | 1 | + (WHO II) | — (D) | + |

? Not applicable or Not available
— Absent
Numbers in Bold are non-degraders
1 Proteinuria positive

### Example 15

### ELISA for the Detection of NET(s) in Fluid Body Samples

### Coating of Capture Antibody:

An ELISA plate (Maxisorp from Nunc) was incubated over night at room temperature with diluted PL2-3 mouse monoclonal antibody (directed against nucleosome complex; (Losman et al., J Immunol (1992), 148:1561-1569)). The PL2-3 mouse monoclonal antibody was diluted in carbonate buffer (100 mM, pH 9.5-9.7) to a final concentration of 4 μg/ml. Subsequently, the cavities of the 96 well plates were carefully emptied.

### Blocking:

Then, 200 μl blocking buffer (PBS+1% BSA+0.01% Tween 20) was added to each well and incubated for 60 min at 37° C. Subsequently, cavities were carefully emptied and stored at −196° C.

### Addition of Human Fluid Samples

After thawing the plates, samples were diluted in blocking buffer (1:10 in master plate) and 50 μl of each, blocking buffer and diluted sample from master plate (final dilution 1:20), was added to cavities of ELISA plate and incubated for 60 min at 37° C. 3 steps of washing with washing buffer (phosphate buffer: 10 mM, pH 7.2-7.4/0.1% v/v Tween 20/300 mM NaCl) followed.

### Detection of NET(s):

First, 100 μl rabbit anti-Neutrophil Elastase polyclonal antibody was added (Calbiochem 481001 in blocking buffer, final concentration 12 μg/ml) and incubated for 60 min at 37° C. 3 steps of washing with washing buffer (phosphate buffer: 10 mM, pH 7.2-7.4/0.1% v/v Tween 20/300 mM NaCl) followed.

### Secondary Antibody:

Second, 100 μl donkey anti-rabbit antibody coupled to POD (Jackson Immuno Research 711-035-152 1:500 in blocking buffer) was added and incubated for 30 min at 37° C. 3 steps of washing with washing buffer (phosphate buffer: 10 mM, pH 7.2-7.4/0.1% v/v Tween 20/300 mM NaCl) followed.

### Signal Intensification:

100 μl rabbit anti-POD coupled to POD (Sigma P-2026 1:500 in blocking buffer) was added and incubated for 30 min at 37° C. 3 steps of washing with washing buffer (phosphate buffer: 10 mM, pH 7.2-7.4/0.1% v/v Tween 20/300 mM NaCl) followed. Finally, 100 μl substrate in substrate buffer (0.5 ml TMB 1 mg/ml in DMSO+4.5 ml 50 mM phosphate/citrate buffer+5 μl H₂O₂) was added and incubated for 10 min at 37° C. Extinction was read at 650 nm.

### Example 16

### ELISA for Measuring NET Degradation by Fluid Body Samples

### Coating of Capture Antibody:

An ELISA plate (Maxisorp from Nunc) was incubated for 30 min at 37° C. with diluted PL2-3 mouse monoclonal antibody (directed against nucleosome complex (H2A+H2B+DNA); (Losman et al., J Immunol (1992), 148:1561-1569)). The PL2-3 mouse monoclonal antibody was diluted in carbonate buffer (100 mM, pH 9.5-9.7) to a final concentration of 2 μg/ml. 3 steps of washing with washing buffer (phosphate buffer: 10 mM, pH 7.2-7.4/0.1% v/v Tween 20/300 mM NaCl) followed.

### Blocking:

Subsequently, 200 μl per well of blocking buffer (PBS+1% BSA+0.01% Tween 20) were added and incubated for 30 min at 37° C. 3 steps of washing with washing buffer (phosphate buffer: 10 mM, pH 7.2-7.4/0.1% v/v Tween 20/300 mM NaCl) followed.

### Addition of NET(s):

NET(s) (supernatant of stimulated human neutrophils; see Example 3 hereinabove) were added at a concentration of 50 μg DNA/ml in 50 μl blocking buffer (PBS, 1% BSA, 0.01% Tween 20) and incubated for 30 min at 37° C. 3 steps of washing with washing buffer (phosphate buffer: 10 mM, pH 7.2-7.4/0.1% v/v Tween 20/300 mM NaCl) followed.

### Addition of Human Serum Samples (SLE/Normal Donors):

Samples were diluted in blocking buffer (1:10 to 1:1000), 50 μl thereof added to cavities of ELISA plate and incubated for 60 min at 37° C. 3 steps of washing with washing buffer (phosphate buffer: 10 mM, pH 7.2-7.4/0.1% v/v Tween 20/300 mM NaCl) followed.

### Detection of NET(s):

100 μl rabbit anti-Neutrophil Elastase (Calbiochem 481001 in blocking buffer, final concentration 12 μg/ml) was added and incubated for 60 min at 37° C. 3 steps of washing with washing buffer (phosphate buffer: 10 mM, pH 7.2-7.4/0.1% v/v Tween 20/300 mM NaCl) followed.

### Signal Intensification

100 μl rabbit anti-POD coupled to POD (Sigma P-2026, 1:500 in blocking buffer) was added and incubated for 30 min at 37° C. 3 steps of washing with washing buffer (phosphate buffer: 10 mM, pH 7.2-7.4/0.1% v/v Tween 20/300 mM NaCl) followed. Finally, 100 μl substrate in substrate buffer (0.5 ml TMB 1 mg/ml in DMSO+4.5 ml 50 mM phosphate/citrate buffer+5 μl H₂O₂) was added and incubated for 10 min at 37° C. Extinction was read at 650 nm.

### Example 17

### Identification of NET Components

Acetone precipitation of NET components

1.5×10⁶ polymorphonuclear granulocytes (PMN)/well (12-well plates) were seeded in RPMI HEPES 10 mM and PMA 20 nM and incubated for 3 h at 37° C. in 5% CO₂. 3 steps of washing in 1 ml of pre-warmed RPMI HEPES followed (to take away all the supernatant) and then 1 ml RPMI HEPES was added. Subsequently, 4 U MNase per well was added and incubated for 20 min at 37° C. The reaction was stopped by adding 2 mM EDTA. The supernatant was collected and centrifuged at 300 g for 10 min. The supernatant was again collected and centrifuged at 16000 g for 20 min. The supernatant was then supplemented with 4 volumes of ice-cold acetone and incubated at 4° C. over night. The mixture was centrifuged twice at 9000 g for 30 min in siliconized Eppendorf reaction tubes (after each centrifugation, 1 ml was discarded and 1 ml of the precipitate added). As an intermediate steps to check the NET(s) were formed by PMA activation, 2 μM of Sytox® green was added and stained for NET-DNA. This allows to check if the neutrophils were activated and eventually formed NET(s). 2 μM Sytox® green was also used after the NET(s) were treated with MNase as mentioned above, to check if there is any NET-DNA left. There should not be a prominent staining after MNase, since MNase digests NET(s) and releases into the supernatant.

### Example 18

### ELISA for Measuring the Degradation of Neutrophil Extracellular Traps by Human Serum Samples

The specificity of the NET ELISA was confirmed as shown in FIG. 12 and as described herein below. NET were generated from neutrophils isolated from a healthy donors and processed them in the NET ELISA as described above. As shown in FIG. 12, NET were detected by the ELISA (the mean of the OD is set forth under each column). If the NET were degraded with purified DNase1 which completely degrades NET (as demonstrated in Hakkim et al., Proc Nat Acad Sci USA (2010), 107: 9813-9818), no signal was obtained. Micrococcal nuclease (MNase) cuts NET into pieces, but does not degrade them completely. Thus, NET treated with MNase are also detected by the NET ELISA. As controls, the cytosol and the nucleus of Hela-cells were separated. Neither of them is recognized by the NET ELISA. Since the nucleus of a HeLa cell contains chromatin, it was probably retained by the first antibody, but the ELISA did not detect them as this chromatin does not contain Neutrophil Elastase.

### Preparation of ELISA-Plates: Coating of Capture Antibody

First, Streptavidin (Biolabs N7021S) was diluted in carbonate buffer (100 mM, pH 9.5-9.7) to a final concentration of 2 μg/ml and incubated for 60 min at 37° C., followed by 3× washing with PBS+0.01% Tween 20. Then, biotinylated PL2-3 mouse monoclonal antibody (directed against nucleosome complex (H2A+H2B+DNA), Losman et al., J Immunol (1992), 148:1561-1569) was diluted in PBS and incubated for 60 min at 37° C.

### Blocking

250 μl blocking buffer (PBS+1% BSA+0.01% Tween 20) was added per well and incubated for 60 min at 37° C. Plates were frozen at −80° C.

### NET Degradation

NET (supernatant of stimulated human neutrophils, DNA content 10 ng/μl; prepared as described herein) at a 1:20 solution were contacted with human sera 1:20 in blocking buffer. Undigested NET 1:20 in blocking buffer (no serum) was used as control. This step was followed by incubation over night at 37° C. Next, plates were thawed and remaining blocking buffer was discarded.

### Addition of Human Serum Samples (Overnight Degradation)

NET/serum samples were added after overnight incubation, 100 μl per cavity, and incubated for 60 min at 37° C., followed by 4× washing as described above.

### Detection of NET

100 μl rabbit anti-human Neutrophil Elastase (Calbiochem 481001, 1 μg/ml in blocking buffer) was added and incubated for 60 min at 37° C., followed by 4× washing as described above.

### Secondary Antibody

Donkey anti-rabbit IgG coupled to POD (Jackson Immunolab 711-035-152, 1:2000 in blocking buffer) was incubated for 30 min at 37° C. and washed 4× as described above.

### Signal Intensification

100 μA rabbit anti-POD coupled to POD (Sigma 1291, 1:1000 in blocking buffer) was added and incubated for 30 min at 37° C., followed by 4× washing as described above. Next, 100 μl TMB substrate (BD OptEIA) was added and incubated for 10 min at 37° C. The reaction was stopped with 50 μl 2N H₂SO₄, the extinction was read at 450 nm. The OD of control sample (NET without serum) was set as 100%. The OD of test sera was expressed as percentage of control. Sera with less than 60% degradation (ELISA OD >60% of NET control) were considered predictive for renal manifestations.

### Example 19

### Sensitivity of NET ELISA

To test the sensitivity of the NET ELISA, NET derived from neutrophils were isolated from a healthy volunteer as described and the amount indicated in FIG. 13 was added to the ELISA. It was show that this method is capable to detect concentrations as low as 10 ng NET-DNA per 100 μl. The curve was in a linear range at least until 300 ng/100 μl. This demonstrates that the NET ELISA is quantitative.

### Example 20

### Reduced Degradation of NET by Some SLE Sera

NET were generated from a healthy donor as described and then incubated with serum from 34 different SLE patients overnight. A control where NET were incubated with buffer (no serum) was considered as NET input and set to 100% NET(s).

Many sera degraded NET; cf. FIG. 14. For example, the serum of patient 2 degraded most of the NET: when the remaining NET were measured, only 20% of the NET were left. Threshold was set at 60% (Hakkim et al., Proc Nat Acad Sci USA (2010), 107: 9813-9818). Sera from patients #13, 25 and 29 was shown to have low NET degradation capability. This means that even after an overnight incubation, more than 60% of the NET remain in the dish. These patients have a higher risk of developing renal complications. The percentage is similar to the one described in Hakkim et al., Proc Nat Acad Sci USA (2010), 107: 9813-9818, although in that publication % NET degradation was reported, while % NET remaining is presented. Yet, as described in context with the present invention, these two measurements can be use analogously.

1. In vitro method for identifying an increased risk of a systemic lupus erythematosus (SLE) patient for developing renal manifestations wherein the method comprises:
(a) obtaining a sample of body fluid from said patient;
(b) contacting said sample with one or more components of neutrophil extracellular traps (NET);
(c) incubating said sample contacted with said NET, thereby allowing the NET to be degraded;
(d) isolating non-degraded NET and degraded NET separately;
(e) determining NET degradation level by measuring either
(i) the amount of one or more selected NET components present in non-degraded NET; or
(ii) the amount of one or more selected NET components released from degraded NET; and
(f) (α) comparing the result with a control where the NET were contacted with a sample of body fluid obtained from a healthy individual, or
(β) comparing the ratio of degraded NET versus the total NET within one
sample,
wherein
(α) a NET degradation level of the patient sample compared to the control sample of less than 70% or
(β) a NET degradation level of degraded NET compared to the total NET of less than 70%
indicates an increased risk of the patient for developing renal manifestations.

2. The method of claim 1, wherein (a) a NET degradation level of the patient
sample compared to the control sample of less than 60% or (β) a NET degradation level of degraded NET compared to the total NET of less than 60% indicates an increased risk of a systemic lupus erythematosus (SLE) patient for developing renal manifestations.

3. The method of claim 1, wherein the NET is derived from neutrophils of healthy donors.

4. The method of claim 1, wherein the NET is artificial NET.

5. The method of claim 1, wherein the NET is immobilized on a solid phase.

6. The method of claim 5, wherein the NET is bound directly to the solid phase.

7. The method of claim 6, wherein the NET is bound to the solid phase via a polypeptide binding specifically to a NET component selected from the group consisting of nucleosome complex, DNA, Histone H2A, Histone H2B, Histone H3, Neutrophil Elastase, Histone H4, S100A8, Lactoferrin, Azurocidin, Cathepsin G, S100A9, Myeloperoxidase, Proteinase 3, Actin, Lysozyme C and Catalase.

8. The method of claim 7, wherein the NET component is the nucleosome complex comprising Histone H2A, Histone H2B and DNA.

9. The method of claim 7, wherein the NET component is Histone H2A, Histone H2B and/or DNA.

10. The method of claim 7, wherein the NET component is Neutrophil Elastase.

11. The method of claim 7, wherein the polypeptide is an antibody.

12. The method of claim 1, wherein the NET degradation is determined by measuring the amount of one or more selected NET components released from degraded NET.

13. The method of claim 12, wherein the measured NET component released from degraded NET is selected from the group consisting of Neutrophil Elastase, nucleosome complex, DNA, Histone H2A, Histone H2B, Histone H3, Histone H4, S100A8, Lactoferrin, Azurocidin, Cathepsin G, S100A9, Myeloperoxidase, Proteinase 3, Actin, Lysozyme C and Catalase.

15. The method of claim 13, wherein the measured NET component released from degraded NET is DNA.

14. The method of claim 13, wherein the measured NET component released from degraded NET is Neutrophil Elastase.

16. The method of claim 1, wherein the NET degradation is determined by measuring the amount of one or more selected NET components present in non-degraded NET.

17. The method of claim 16, wherein the measured NET component present in non-degraded NET is selected from the group consisting of Neutrophil Elastase, nucleosome complex, DNA, Histone H2A, Histone H2B, Histone H3, Histone H4, S100A8, Lactoferrin, Azurocidin, Cathepsin G, S100A9, Myeloperoxidase, Proteinase 3, Actin, Lysozyme C and Catalase.

19. The method of claim 17, wherein the measured NET component present in non-degraded NET is DNA.

18. The method of claim 17, wherein the measured NET component present in non-degraded NET is Neutrophil Elastase.

20. The method of claim 1, wherein the sample of body fluid is selected from the group consisting of blood, plasma, serum, lymphatic fluid, cerebrospinal fluid, vaginal fluid, semen, sputum, broncho-alveolar lavage fluid, ascites, faeces and faeces extracts.

21. The method of claim 20, wherein the sample of body fluid is blood.

22. The method of claim 20, wherein the sample of body fluid is serum.

23. The method of claim 1, wherein said measuring of the amount of one or more selected NET components in (i) or (ii) is performed with an ELISA.

24.-26. (canceled)

27. A kit comprising:
(a) an antibody binding to the nucleosome complex comprising Histone H2A, Histone H2B and DNA;
(b) an antibody binding to a NET component selected from the group consisting of Neutrophil Elastase, nucleosome complex, DNA, Histone H2A, Histone H2B, Histone H3, Histone H4, S100A8, Lactoferrin, Azurocidin, Cathepsin G, S100A9, Myeloperoxidase, Proteinase 3, Actin, Lysozyme C and Catalase;
(c) whole NET or one or more NET components selected from the group consisting of Neutrophil Elastase, nucleosome complex, DNA, Histone H2A, Histone H2B, Histone H3, Histone H4, S100A8, Lactoferrin, Azurocidin, Cathepsin G, S100A9, Myeloperoxidase, Proteinase 3, Actin, Lysozyme C and Catalase; and
(d) solvents, diluents and/or buffers.

28. The kit of claim 29, further comprising an ELISA plate.
